(19) United States
(12) Patent Application Publication
(10) Pub. No.: US 2025/0000794 A1
(43) Pub. Date: Jan. 2, 2025

(54) IMPROVED DRUG PROCESSING METHODS TO INCREASE DRUG LOADING

(71) Applicant: BOARD OF REGENTS, THE UNIVERSITY OF TEXAS SYSTEM, Austin, TX (US)

(72) Inventors: Robert O. WILLIAMS, III, Austin, TX (US); Daniel A. DAVIS, JR., Austin, TX (US); Stephen Ambrose THOMPSON, Austin, TX (US)

(21) Appl. No.: 18/292,366

(22) PCT Filed: Jul. 27, 2022
(86) PCT No.: PCT/US2022/074178

§ 371 Date: Jan. 26, 2024

Related U.S. Application Data
(60) Provisional application No. 63/203,592, filed on Jul. 27, 2021.

(51) Int. Cl. A61K 9/10; A61K 9/14

(52) U.S. Cl. CPC A61K 9/10; A61K 9/146

(57) ABSTRACT

The disclosure provides for improved methods of formulating pharmaceutical compositions containing weakly basic or weakly acid active pharmaceutical ingredients that are also poorly water-soluble. The methods employ the use of strong bases with weakly acidic APIs and strong acids with weakly basic APIs, thereby improving drug loading.

This application claims the benefit of priority to U.S. Provisional Application No. 63/203,592, filed on Jul. 27, 2021, the entire contents of which are hereby incorporated by reference.

### BACKGROUND

### 1. Field

The present disclosure relates in general to the field of pharmaceutical preparation and manufacturing, and more particularly, pharmaceutical formulations of poorly soluble drugs that are also weakly basic or acidic using strong acids or strong bases, respectively.

### 2. Description of Related Art

The majority of new active pharmaceutical ingredient (API) candidates are poorly-water soluble (PWS); this can cause their oral bioavailability to be insufficient for therapeutic use. Because the bioavailability of these candidates is low, a comparatively large quantity of API must be ingested to achieve clinical effects. This large quantity of API can require multiple large pills or tablets at frequent (e.g., >1 time per day) intervals that may cause undue burden to patients already suffering from health-related issues. There is a need for reducing pill burden, which can worsen outcomes of patients if they are unable to follow medication regimens, by increasing the oral bioavailability of these API candidates. There are a variety of processes that have been used to improve the water solubility of PWS API. One of these methods is to formulate the API into an amorphous solid dispersion (ASD) where the crystalline structure of the API is disrupted and stabilized in a polymeric carrier. This disruption of the structure is energetically unfavorable and leads to an increased solubility of the ASD as compared to the comparably energetically favorable crystalline state. Hot melt extrusion (HME) is a common processing method for formulating these APIs into ASDs and uses thermal and mechanical energy to convert the crystalline API to amorphous and dissolve it in the polymeric carrier and the other excipients. HME is a useful processing method because it is comparatively easy to scale and is a continuous manufacturing process. In addition, this method does not require the use of solvents to formulate, in contrast with spray-drying, and so does not require additional drying steps when used with compounds that need to be protected from water. According to Maa et al., reducing residual moisture content below 3% w/w is difficult using only spray-drying (10). ASD particle size formulated using HME tends to be smaller than those formulated by spray-drying and this can be advantageous for moisture-sensitive material as smaller particle size reduces the rate of water sorption (9).

While this technique has been in use for several decades, there are still limitations to its use, particularly for drugs with high melting points and those that degrade when exposed to thermal or mechanical energy. Another limitation is that it can be difficult to achieve high drug load (w/w) ASDs using HME. Drug loading (DL) is an issue and challenge for APIs that require high dosing as, if high drug loading cannot be achieved, patients may be required to take multiple large pills or tablets which can reduce compliance and cause worse patient outcomes. The maximum drug loading for an ASD is dependent on the miscibility (i.e., ability to be dissolved) of API in the polymeric carrier. This miscibility of the API in polymer is dependent on a variety of factors and is frequently temperature dependent. While ASDs can be formulated above the solubility of API in a particular polymer, they tend to be less physically stable (metastable) and the amorphous API can revert to its crystalline form. Not only do the polymers used in ASDs act as carriers and stabilizers for the amorphous API, they also can improve the dissolution profile of the API when in the gastro-intestinal (GI) tract and thus can improve the drug's bioavailability.

KinetiSol dispersing (KSD) is an example of a high energy, fusion-based process that does not require an external heat source to process drug-polymer blends into an ASD. KSD operates at several orders of magnitude greater than HME and has typical processing times of <30 seconds. This process is still limited to the drug's miscibility in the polymer at the processing temperature; therefore, increasing the drug's miscibility to enable higher drug loading or decrease the processing temperature would be beneficial for thermally labile PWS API.

For ASDs comprising thermally-labile API, the challenge of achieving high drug loading is heightened as one technique available for improving the miscibility of the API in polymeric carrier is to increase the temperature of processing. While the increase in processing temperature improves the solubility of the API in polymer, it can also cause the degradation of the API or polymer if it is too high. In some cases, the range of processing parameters, or design space, that results in the successful conversion of API to amorphous form but does not result in degradation of the polymer or API can be small or even non-existent. For this reason, there is a need to expand the design space of these hard-to-formulate APIs to improve drug loading.

### SUMMARY

Thus, in accordance with the present disclosure, there is provided a method of preparing a pharmaceutical composition comprising an amorphous solid dispersion (ASD) of a poorly water-soluble active pharmaceutical ingredient (API), wherein said API is a weak base or weak acid, comprising (a) providing an API that is a weak base or weak acid; (b) contacting said API with a polymeric carrier and:
(i) a strong base when said API is a weak acid; or
(ii) a strong acid when said API is a weak base; and
   (c) processing the mixture of step (b) to produce an ASD. The API may exhibit a high melting point, may be thermally sensitive, may be shear sensitive, and/or may exhibit pH dependent solubility with thermal and/or high energy processing. Processing may comprise a high energy mixing process, such as a high energy fusion process (e.g., KinetiSol® Dispersing), or a thermal process, such as hot melt extrusion. The water solubility of the API may be less than 1 mg/mL when measured using high pressure liquid chromatography (HPLC) at 20° C. The API may be telmisartan, itraconazole, meloxicam, diclofenac, or atazanavir.

The strong base may be NaOH, KOH, Ca(OH)₂, or LiOH. The strong base of sodium hydroxide or potassium hydroxide may be formed in situ from sodium carbonate or potassium carbonate, such as where the strong based is sodium hydroxide produced from Na₂CO₃ from CO₂ escape during extrusion. In some embodiments, steps (b) and (c) may be performed simultaneously. The strong base may be a water-soluble strong base. The strong acid may be hydrochloric acid, chloric acid, sulfuric acid, hydrobromic acid, nitric acid, or P-toluene sulfonic acid. The strong acid may be a water-soluble strong acid.

The mixture of step (b) may comprise 0.1-15% (w/w) strong base or strong acid and/or polymer present in said pharmaceutical composition at about 1% to 50% (w/w). The polymeric carrier may be water soluble, e.g., Soluplus®, methylcellulose, ethylcellulose, hydroxymethylcellulose, hydroxyethylcellulose, hydroxypropylcellulose, hydroxybutylcellulose, hydroxyethyl methylcellulose, hydroxypropyl methylcellulose, carboxymethylcellulose, hydroxypropyl methylcellulose acetate succinate, cellulose acetate phthalate, cellulose acetate trimellate, hydroxypropyl methylcellulose phthalate, cellulose acetate butyrate, polyvinylpyrrolidones, polyacrylates, polymethacrylates, polyvinyl alcohols, polyethylene glycols, polyvinyl acetate polyvinylpyrrolidone copolymers, polyethylene oxides, dimethylaminoethyl methacrylate-methacrylic acid ester copolymers, ethylacrylate-methylmethacrylate copolymers, poly(methyacrylate ethylacrylate) copolymers, poly(methacrylate methylmethacrylate) copolymers, starches, pectins, polysaccharides, gum arabic, guar gum, and xanthan gum. The pharmaceutical composition may not contain a plasticizer and/or may not contain a processing agent. The pharmaceutical composition may comprise a surfactant.

The pharmaceutical composition may have an increase in drug loading compared to a pharmaceutical composition prepared by a comparable method without said strong acid or strong base, such as an increase in drug loading of about 2.5% to about 50%. The pharmaceutical composition may comprise less than about 1% degradation products of said API. The ratio of API to polymeric carrier may be about 3:2, 5:4, 1:1, 2:3, 1:2, 1:3, 1:4, or 1:5. Step (b) may be performed at a temperature of about 100° C., about 125° C., about 150° C., about 175° C., about 200° C., about 220° C., or about 100° C. to about 220° C.

The method of claims 1-18, wherein said pharmaceutical composition comprises a second API, such as compositions containing two or more API that are used to treat hypertension (e.g., ACE inhibitors such as lisinopril, enalapril, benazepril, captopril; beta-blockers such as metoprolol, carvedilol, esmolol, nebivolol; calcium channel blockers such as diltiazem, verapamil, amlodipine, nifedipine; diuretics including thiazide diuretics such as hydrochlorothiazide and chlorthalidone or furosemide, spironolactone, bumetanide, torsemide or others), diabetes (e.g., metformin; SGLT2 inhibitors including empagliflozin, dapagliflozin, canagliflozin; DDP4 inhibitors including sitagliptin, saxagliptin, linagliptin; GLP-1 agonists including dulaglutide, exenatide, semaglutide; and others), or cancer (e.g., chlorambucil, lomustine, tobrozole and echinomycin).

Also provided is a pharmaceutical composition prepared according to a method as described herein.

Although making and using various embodiments of the present disclosure are discussed in detail below, it should be appreciated that the present disclosure provides many inventive concepts that may be embodied in a wide variety of contexts. The specific aspects and embodiments discussed herein are merely illustrative of ways to make and use the disclosure, and do not limit the scope of the disclosure.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: PXRD of extrudate and physical mixtures containing TEL and Soluplus® processed at 180° C. and 100 RPM on a MiniHaake extruder. The presence of peaks at any of 6.8, 13, 15, and 23° 2-theta are indicative of crystalline TEL.

FIG. 2: DSC of physical mixtures and extrudates containing TEL and Soluplus®. An endothermic event occurring either broadly between 220-260° C. or at 270° C. was indicative of crystalline TEL.

FIG. 3: PXRD of extrudate and physical mixtures containing TEL, base, and Soluplus® processed at 180° C. and 100 RPM on a MiniHaake extruder. The presence of peaks at any of 6.8, 13, 15, and 23° 2-theta are indicative of crystalline TEL.

FIG. 4: PXRD of extrudate and physical mixtures containing NaOH and Soluplus® processed at 180° C. and 100 RPM on a MiniHaake extruder. The presence of peaks at any of 6.8, 13, 15, and 23° 2-theta are indicative of crystalline TEL.

FIG. 5: PXRD of extrudate and physical mixtures containing TEL, Arginine, and Soluplus® processed at 200° C. and 100 RPM on a MiniHaake extruder. The presence of peaks at any of 6.8, 13, 15, and 23° 2-theta are indicative of crystalline TEL.

FIG. 6: PXRD of extrudate and physical mixtures containing TEL, sodium carbonate (Na₂CO₃), and Soluplus® processed at 200° C. and 100 RPM on a MiniHaake extruder. The presence of peaks at any of 6.8, 13, 15, and 23° 2-theta are indicative of crystalline TEL.

FIG. 7: PXRD of extrudate and physical mixtures containing TEL, calcium hydroxide (Ca(OH)₂), and Soluplus® processed at 200° C. and 100 RPM on a MiniHaake extruder. The presence of peaks at any of 6.8, 13, 15, and 23° 2-theta are indicative of crystalline TEL.

FIG. 8: PXRD of extrudate and physical mixtures containing TEL, sodium hydroxide (NaOH), and Soluplus® processed at 200° C. and 100 RPM on a MiniHaake extruder. The presence of peaks at any of 6.8, 13, 15, and 23° 2-theta are indicative of crystalline TEL.

FIG. 9: PXRD of extrudate and physical mixtures containing TEL, potassium hydroxide (KOH), and Soluplus® processed at 200° C. and 100 RPM on a MiniHaake extruder. The presence of peaks at any of 6.8, 13, 15, and 23° 2-theta are indicative of crystalline TEL.

FIG. 10: PXRD of extrudate and physical mixtures containing TEL, lithium hydroxide (LiOH), and Soluplus® processed at 180° C. and 100 RPM on a MiniHaake extruder. The presence of peaks at any of 6.8, 13, 15, and 23° 2-theta are indicative of crystalline TEL.

FIG. 11: PXRD of material either containing TEL, potassium hydroxide (KOH), and Soluplus (F1), or TEL and Soluplus (F2) processed at 5500 RPM for 30 seconds with an ejection temperature of 180 C processed on a Kinetisol Solid Dispersion formulator. The presence of peaks at any of 6.8, 13, 15, and 23° 2-theta are indicative of crystalline TEL and the region at 6.8° 2-theta is highlighted for visual clarity.

FIG. 12: DSC of physical mixtures and extrudates containing TEL, potassium hydroxide and Soluplus® (30:7:63 w/w). An endothermic event occurring either broadly between 220-260° C. or at 270° C. was indicative of crystalline TEL.

FIG. 13: PXRD of extrudate and physical mixtures containing TEL, potassium hydroxide (LiOH), and Soluplus® (30:7:63 w/w) processed at 150° C., 160° C., 170° C. or 180° C. and at 100 RPM on a MiniHaake extruder. The presence of peaks at any of 6.8, 13, 15, and 23° 2-theta are indicative of crystalline TEL.

FIG. 14: PXRD of extrudate and physical mixtures containing meloxicam, potassium hydroxide (KOH) and Soluplus® processed at 140° C. and 100 RPM on a MiniHaake extruder. The presence of peaks is indicative of crystalline meloxicam.

### DETAILED DESCRIPTION

As discussed above, the issue of unacceptable bioavailability of drugs that exhibit unacceptable (e.g., low) drug loading in amorphous solid dispersions remains a significant problem in pharmaceutical formulation. Here, the inventors demonstrate that they solved the issue of unacceptable bioavailability of certain drugs due to low drug loading in amorphous solid dispersions. Especially, this invention is advantageous with drugs that have high melting points, are thermally or shear sensitive, and exhibit low and pH dependent solubility by thermal and/or high energy mixing processing. Previously, drugs that exhibit these characteristics (e.g., telmisartan) have been formulated as ASDs using solvent evaporation methods such as spray-drying (1). Solvent evaporation methods are unfavorable in manufacturing due to the use of either organic solvent that are hazardous to human health and must be removed by multiple and expensive drying steps, or else aqueous solutions that can result in substantial residual water (up to ˜8% w/w) that can negatively impact the stability of ASDs (1, 9). In one example, Maa et al., attempted to reduce the 7.2% residual water present in a spray-dried protein-mannitol formulation, by vacuum drying the powder (10). They were able to reduce the moisture content to 2.8%, but despite this, upon exposure to ambient humidity for 2 hours, the moisture content rapidly increased to 6.9% and after subsequent storage in a sealed vial, the moisture content increased above the spray-dried formulation that did not undergo vacuum drying to 7.4% vs. 7.2%, respectively. This indicates that even after spray-dried formulations are dried, they can be susceptible to moisture sorption, especially when comprised of hygroscopic materials.

Specifically, poorly water-soluble drugs like telmisartan, a weakly acidic drug, formulated as ASDs are generally prepared by solvent evaporation of an alkaline aqueous solution (telmisartan is soluble in water only under alkaline conditions). The marketed product Micardis® (telmisartan) is formulated in this way. Giri et al. showed that under accelerated stability conditions (40° C./75% RH) the residual moisture content of Micardis® tablets increases from 2.13% (+/−0.57%) to approximately 13% after only two weeks, a nearly 6× increase (8). This contrasts with a similar HME formulation comprising telmisartan, sodium carbonate, and Soluplus® that reached only 5.0% residual moisture content under identical conditions after 4 weeks, from an initial 2.89%. The higher moisture content in the Micardis® tablet was associated with a substantial 40% drop in potency as compared to an only 5% drop in potency of the HME formulation after 4 weeks. This study supports the observation made by Haser et al., that spray-dried (and those similarly prepared by other solvent evaporation methods) particles have smaller particle sizes (i.e., larger specific surface area) that result in an increased rate of moisture absorption that can cause instability of amorphous materials as compared to those formulated by HME (9).

A recent report from Almotairy et al. (2021) disclosed formulated ASDs of telmisartan and hydroxypropyl methylcellulose acetate succinate L grade (HPMCAS LG) using HME with meglumine, sodium carbonate or Neusilin S2 to modify the microenvironment pH during in vitro dissolution (11). HPMCAS is a cellulosic polymer with both succinic and acetic acid moieties present that make it weakly acidic and because of the high viscosity of HPMCAS LG at their chosen extrusion temperature, the authors were unable to extrude except when adding a plasticizer at a 10% (w/w) loading relative to the polymer. In addition, they state that they were unable to achieve a >10% (w/w) drug loading ASD using their polymer. Their 10% (w/w) DL formulations containing 2.5-10% (w/w) sodium carbonate or 10% (w/w) meglumine were also stated to be partially crystalline by XRD. The authors did not speculate as to why this occurred, but it is reasonable that with a weakly acidic polymer like HPMCAS, the use of a small percentage of weak base would not be sufficient to alter the miscibility of telmisartan in the polymer and that either a greater percentage or stronger base would be required. The in vitro dissolution of these formulations was tested in pH 6.8 buffer with 0.05% SLS. The best performing formulation contained 10% w/w meglumine and achieved an approximate 5× supersaturation as compared to pure telmisartan. As compared to these experiments the benefit of utilizing a strong base with a weakly acidic API during processing by HME is apparent as the drug loading can be increased from a maximum ˜10% w/w to at least 50%. In addition, because the techniques described in this patent cause complete, as opposed to partial, conversion to amorphous API, the supersaturation of API achieved as compared to the physical mixture was 25× relative to the only 5× achieved here.

This disclosure reports improved drug loading and bioavailability of ASDs comprising drugs that exhibit high melting point (e.g., >˜160° C.), thermal sensitivity, shear sensitivity, and/or those that have low and pH dependent solubility by thermal and/or high energy mixing processing in an amorphous solid dispersion. Specifically, this is achieved by pH modification of drug-polymer system during HME to obviate the need for solvent (e.g., spray-drying) methods otherwise required to form amorphous ASDs comprising basic or acidic API. By use of this method to produce these ASDs, a high drug loading can be achieved that avoids the issues described above with formulations produced by solvent methods. Specifically, by the addition of amounts (e.g., <10% w/w) of a strong base/acid to the premix, including formation of the strong base/acid in situ, that is then extruded, the loading of drugs that have pH dependent solubility in water can be increased to at least 30% and more than 50% in ASDs. By improving the drug loading of the ASD, greater flexibility can be achieved in dosage form formulation for improved performance or patient acceptability.

These and other aspects of the disclosure are discussed in detail below.

### I. Definitions

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity but include the general class of which a specific example may be used for illustration.

Regarding the values or ranges recited herein, the term “about” is intended to capture variations above and below the stated number that may achieve substantially the same results as the stated number, including for example, +/−10% for weight or volume measurements. In the present disclosure, each of the variously stated ranges is intended to be continuous so as to include each numerical parameter between the stated minimum and maximum value of each range. For example, a range of about 1 to about 4 includes about 1, 1, about 2, 2, about 3, 3, about 4, and 4. The terminology herein is used to describe specific embodiments of the disclosure, but their usage does not delimit the disclosure, except as outlined in the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “heterogeneously homogenous composite” refers to a material composition having at least two different materials that are evenly and uniformly distributed throughout the volume.

As used herein, the phrase “reference standard active pharmaceutical ingredient” means the most thermodynamically stable form of the active pharmaceutical ingredient that is currently available.

As used herein, the term “high melt viscosity” refers to melt viscosities greater than about 10,000 Pa*s.

A “high melting point API” refers to a melting point that is greater than about 160° C. as measured by differential scanning calorimetry or other suitable means.

As used herein, the term “thermally labile API” refers to an API that degrades at its crystalline melting point, or one that degrades at temperatures below the crystalline melting point when in a non-crystalline (amorphous) form. As used herein, the term “thermolabile polymer” refers to a polymer that degrades at or below about 200° C.

As used herein, “thermally processed” or “processed thermally” means that components are processed by hot melt extrusion, melt granulation, compression molding, tablet compression, capsule filling, film-coating, or injection molding.

As used herein, “drug loading” means that the API is miscible with the polymer composition, i.e., not suspended, and refers to the amount of API relative to the total amount of the composition including inactive ingredients. For example, drug loading of 30% means that the composition contains API at 30% by weight relative to the entire composition that includes inactive ingredients.

As used herein, “extrusion” is the well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders. Furthermore, the extrusion can be carried out through melt extrusion. Components of the present disclosure can be melted and extruded with a continuous, solvent free extrusion process, with or without inclusion of additives. Such processes are well-known to skilled practitioners in the art.

As used herein, “spray congealing” is a method that is generally used in changing the structure of materials, to obtain free flowing powders from liquids and to provide pellets. Spray congealing is a process in which a substance of interest is allowed to melt, disperse, or dissolve in a hot melt of other additives, and is then sprayed into an air chamber wherein the temperature is below the melting point of the formulation components, to provide congealed pellets. Such a process is well-known to skilled practitioners in the art.

As used herein, “solvent dehydration” or “spray drying technique” is commonly employed to produce a dry powder from a liquid or slurry by rapidly drying with a hot gas. This is one preferred method of drying many thermally sensitive materials such as foods and pharmaceuticals. Water or organic solvent-based formulations can be spray dried by using inert process gas, such as nitrogen, argon and the like. Such a process is well-known to skilled practitioners in the art.

As used herein, “bioavailability” is a term meaning the degree to which a drug becomes available to the target tissue after being administered to the body. Poor bioavailability is a significant problem encountered in the development of pharmaceutical compositions, particularly those containing a drug that is not highly soluble.

As used herein, the phrase “pharmaceutically acceptable” refers to molecular entities, compositions, materials, excipients, carriers, and the like that do not produce an allergic or similar untoward reaction when administered to humans in general.

As used herein, “poorly water soluble” or “PWS” means an API that has a solubility such that more than 30 parts of solvent are required to dissolve one part of the API. A POSA would understand that “poorly water soluble” means sparingly soluble, slightly soluble, very slightly soluble and practically insoluble. These are USP descriptive solubility terms defined in the USP; see United States Pharmacopoeia (2021), section titled “Description and Relative Solubility.”

As used herein, “water soluble” means that less than 30 parts of water are required to dissolve one part of solute (from USP, same as above).

A “weak base” is defined herein as a basic substance that does not completely dissociate into their constituent ions when dissolved in solution. Non-limiting examples include ammonia, trimethylamine, pyridine, sodium carbonate; sodium bicarbonate, meglumine, arginine, and lysine.

In contrast, a “strong base” as used herein is defined as one that will completely dissociate into its constituent ions when dissolved in solution. Non-limiting examples of strong bases include sodium hydroxide, lithium hydroxide, potassium hydroxide, magnesium hydroxide, and calcium hydroxide.

A “weak acid” as used herein is defined as an acidic substance that does not completely dissociate into their constituent ions when dissolved in solution. Non-limiting examples include citric acid, benzoic acid, formic acid, oxalic acid, and acetic acid.

In contrast, a “strong acid” as used herein is defined as one will completely dissociate into its constituent ions when dissolved in solution. Non-limiting examples of strong acids include hydrochloric acid, chloric acid, sulfuric acid, hydrobromic acid, nitric acid, and P-toluene sulfonic acid.

As used herein, “derivative” refers to chemically modified inhibitors or stimulators that still retain the desired effect or property of the original drug. Such derivatives may be derived by the addition, removal, or substitution of one or more chemical moieties on the parent molecule. Such moieties may include, but are not limited to, an element such as a hydrogen or a halide, or a molecular group such as a methyl group. Such a derivative may be prepared by any method known to those of skill in the art. The properties of such derivatives may be assayed for their desired properties by any means known to those of skill in the art. As used herein, “analogs” include structural equivalents or mimetics.

The solution agent used in the solution can be aqueous such as water, one or more organic solvents, or a combination thereof. When used, the organic solvents can be water miscible or non-water miscible. Suitable organic solvents include but are not limited to ethanol, methanol, tetrahydrofuran, acetonitrile, acetone, tert-butyl alcohol, dimethyl sulfoxide, N,N-dimethyl formamide, diethyl ether, methylene chloride, ethyl acetate, isopropyl acetate, butyl acetate, propyl acetate, toluene, hexanes, heptane, pentane, and combinations thereof.

By “immediate release” is meant a release of an API promptly upon administration to an environment over a period of seconds to no more than about 30 minutes once release has begun and release begins within no more than about 2 minutes after administration. An immediate release does not exhibit a significant delay or prolongation in the release of drug.

By “rapid release” is meant a release of an API to an environment over a period of 1-59 minutes or 0.1 minute to three hours once release has begun and release can begin within a few minutes after administration or after expiration of a delay period (lag time) after administration.

As used herein, the term “extended release” profile assumes the definition as widely recognized in the art of pharmaceutical sciences. An extended release dosage form will release an API at a substantially constant rate over an extended period of time or a substantially constant amount of API will be released incrementally over an extended period of time. An extended release tablet generally effects at least a two-fold reduction in dosing frequency as compared to the API presented in a conventional dosage form (e.g., a solution or rapid releasing conventional solid dosage forms).

By “controlled release” is meant a release of an API to an environment over a period of about eight hours up to about 12 hours, 16 hours, 18 hours, 20 hours, a day, or more than a day. By “sustained release” is meant an extended release of an active agent to maintain a constant drug level in the blood or target tissue of a subject to which the device is administered.

The term “controlled release”, as regards to drug release, includes the terms “extended release,” “prolonged release,” “sustained release,” or “slow release,” as these terms are used in the pharmaceutical sciences. A controlled release can begin within a few minutes after administration or after expiration of a delay period (lag time) after administration.

A “slow release dosage form” is one that provides a slow rate of release of API so that API is released slowly and approximately continuously over a period of 3 hours, 6 hours, 12 hours, 18 hours, a day, 2 or more days, a week, or 2 or more weeks, for example.

The term “mixed release” as used herein refers to a pharmaceutical agent that includes two or more release profiles for one or more active pharmaceutical ingredients. For example, the mixed release may include an immediate release and an extended release portion, each of which may be the same API or each may be a different API.

A “timed release dosage form” is one that begins to release an API after a predetermined period of time as measured from the moment of initial exposure to the environment of use.

A “targeted release dosage form” generally refers to an oral dosage form that is designed to deliver an API to a particular portion of the gastrointestinal tract of a subject. An exemplary targeted dosage form is an enteric dosage form that delivers a drug into the middle to lower intestinal tract but not into the stomach or mouth of the subject. Other targeted dosage forms can deliver to other sections of the gastrointestinal tract such as the stomach, jejunum, ileum, duodenum, cecum, large intestine, small intestine, colon, or rectum.

By “delayed release” is meant that initial release of an API occurs after expiration of an approximate delay (or lag) period. For example, if release of an API from an extended release composition is delayed two hours, then release of the API begins at about two hours after administration of the composition, or dosage form, to a subject. In general, a delayed release is opposite of an immediate release, wherein release of an API begins after no more than a few minutes after administration. Accordingly, the API release profile from a particular composition can be a delayed-extended release or a delayed-rapid release. A “delayed-extended” release profile is one wherein extended release of an API begins after expiration of an initial delay period. A “delayed-rapid” release profile is one wherein rapid release of an API begins after expiration of an initial delay period.

A “pulsatile release dosage form” is one that provides pulses of high API concentration, interspersed with low concentration troughs. A pulsatile profile containing two peaks may be described as “bimodal.” A pulsatile profile of more than two peaks may be described as multi-modal.

A “pseudo-first order release profile” is one that approximates a first order release profile. A first order release profile characterizes the release profile of a dosage form that releases a constant percentage of an initial API charge per unit time.

A “pseudo-zero order release profile” is one that approximates a zero-order release profile. A zero-order release profile characterizes the release profile of a dosage form that releases a constant amount of API per unit time.

### II. Methods of Formulating

### A. Hot Melt Extrusion

Hot melt extrusion (HME) is the process of applying heat and pressure to melt a polymer and forcing it through an orifice in a continuous process that is capable for producing polymer products of uniform shape and density. HME has particular relevance to mixing APIs with polymers and has been used to improve various API's bioavailability and can provides product developers of with processing options that maximize API mixing with polymer while minimizing API degradation.

HME is carried out using an extruder—a barrel containing one or two rotating screws that transport material down the barrel. For preparing pharmaceutical formulations, i.e., those that require homogeneous and consistent mixing of multiple ingredients, a twin screw extruder is preferred as the rotation of the inter-meshing screws provides better mixing capable of producing a homogeneous solid of finely dispersed API particles or solid-solution API in polymer. Consistent melt-mixing using twin screw extrusion has been used to improve the dissolution rate and bioavailability of poorly water-soluble API formulations.

Melting is accomplished by frictional heating within the barrel. For a twin-screw extruder, materials undergo shearing between the rotating screws and between the screws and the wall of the barrel as they are conveyed. Additional heating can be generated by barrel-mounted heaters, generally optimized to allow proper mixing and conveyance and to avoid thermal degradation. Twin screw extruders can be engineered to provide different types of mixing and conveying conditions in the different zones present throughout the barrel. Sections of the screw can even be designed to perform particle size reduction, mixing, and conveying functions. Also, choosing a particular length of the screw versus barrel diameter (the L/D ratio) alters the degree of mixing and the number of zones required to achieve the final product characteristics. Rotation of the screws creates distributive and dispersive mixing, the former maximizing the division and recombination of the materials while minimizing energy input by mixing with low extensional and planar-shear effects, while the latter applies extensional and planar shear fields to break the dispersed materials to smaller size, ideally using energy at or slightly above the threshold level needed to break them down.

The use of different mixing elements allows the twin screw extruder to perform both particle size reduction and mixing so that the APIs can be incorporated into the polymer in dispersed form or, if the API solubility in the polymer is high enough, in dissolved form. Since the extrudate cools rapidly on exiting the extruder, any API that is dissolved in the polymer at the mixing temperature may be unable to recrystallize on cooling, leading to supersaturated solid solutions. Different types of exit dies are used to shape the extrudate to the desired profile. Downstream auxiliary components can also be used in the finishing process, including water baths, air knives, conveyor belts, strand-cutters, and spoolers. Pelletizers cut the extrudate into smaller pieces for direct capsule filling, as potential for injection molding.

For most applications, the polymer should be thermoplastic, stable at the temperatures used in the process, and chemically compatible with the API during extrusion. For solid oral dosage forms, water soluble polymers are usually chosen from among well-known polymers already used in pharmaceutical products such as poly(ethylene glycol) and poly(vinylpyrrolidinone).

### B. KinetiSol®

KinetiSol® is an example of a high energy, fusion-based process that utilizes frictional and shear energies to rapidly transition drug-polymer blends into a molten state. Simultaneous to the molten transition, KinetiSol® rapidly and thoroughly mixes the active ingredient with its excipient carrier(s) on a molecular level to achieve a single-phase ASD system. The real-time temperature of the composition within the KinetiSol® chamber is monitored by a computer-control module, and upon reaching the user defined endpoint, molten material is immediately ejected from the process. Total processing times are generally less than 20 seconds, and elevated temperatures are observed for typically less than 5 seconds before discharge and cooling. On a lab-scale, the process is designed to operate in batch mode, whereas in commercial processing, it is operated semi-continuously, achieving product throughput as high as 1,000 kg/hr.

KinetiSol® employs thermokinetic compounding that may be carried out in a thermokinetic chamber using one or multiple speeds during a single, compounding operation on a batch of components to form a pharmaceutical formulation of the present disclosure. A thermokinetic chamber includes a chamber having an inside surface and a shaft extending into or through the chamber. Extensions extend from the shaft into the chamber and may extend to near the inside surface of the chamber. The extensions are often rectangular in cross-section, such as in the shape of blades, and have facial portions. During thermokinetic compounding, the shaft is rotated causing the components being compounded, such as particles of the components being compounded, to impinge upon the inside surface of the chamber and upon facial portions of the extensions. The shear of this impingement causes comminution, frictional heating, or both of the components and translates the rotational shaft energy into heating energy. Any heating energy generated during thermokinetic compounding is evolved from the mechanical energy input. Thermokinetic compounding is carried out without an external heat source. The thermokinetic chamber and components to be compounded are not pre-heated prior to commencement of thermokinetic compounding. The thermokinetic chamber may include a temperature sensor to measure the temperature of the components or otherwise within the thermokinetic chamber.

During thermokinetic compounding, the average temperature of the thermokinetic chamber may increase to a pre-defined final temperature over the duration of the thermokinetic compounding to achieve thermokinetic compounding of the API. The pre-defined final temperature may be such that degradation of the API, polymer, or other components is avoided or minimized. Similarly, the one or multiple speeds of use during thermokinetic compounding may be such that thermal degradation of the API, polymer, or other components is avoided or minimized. As a result, the API, polymer, or other components of the amorphous solid dispersion may lack substantial impurities.

Pressure, duration of thermokinetic compounding, and other environmental conditions such as pH, moisture, buffers, ionic strength of the components being mixed, and exposure to gasses, such as oxygen, may also be such that degradation of any APIs present, one or all excipients, or one or all other components is avoided or minimized. Thermokinetic compounding may be performed in batches or in a semi-continuous fashion, depending on the product volume. When performed in a batch, semi-continuous, or continuous manufacturing process, each thermokinetic compounding step may occur for less than 5, 10, 15, 20, 25, 30, 35, 40, 45, 50, 55, 60, 70, 100, 120, 240, or 300 seconds, inclusive, or for an interval between any of these time points, inclusive, or for an interval between 1 second any any of these time points, inclusive.

Variations of thermokinetic compounding may be used depending on the amorphous solid dispersion and its components. For example, the thermokinetic chamber may be operated at a first speed to achieve a first process parameter, then operated at a second speed in the same thermokinetic compounding process to achieve a final process parameter. In other examples, the thermokinetic chamber may be operated at more than two speeds, or at only two speeds, but in more than two-time internals, such as at a first speed, then at a second speed, then again at the first speed.

With its unique attributes, the KinetiSol® process is providing novel solutions to emerging problems associated with ASD processing. The very brief processing times of KinetiSol® enable production of ASD systems with thermally sensitive APIs and excipients. The high rates of shear inherent to KinetiSol® accelerate solubilization kinetics of drug compounds in molten polymers, which typically results in processing temperatures that are well below the melting point of the API. Consequently, the production of ASD systems with high melting point compounds (>200° C.) in a broad spectrum of concentration enhancing polymers is routinely achieved with KinetiSol®. The KinetiSol® process is not torque limited, and hence processing of highly viscous/non-thermoplastic/high molecular weight polymers can be easily accomplished without the use of plasticizers. The capabilities of KinetiSol® enable the use of unique drug/excipient combinations to create solubility-enhanced compositions that cannot be reproduced or manufactured at large scales by other technologies.

As established technologies in pharmaceutical manufacturing, HME (discussed above) and spray drying are the primary options for most PWS compounds. For molecules with acceptable thermal properties, HME is typically the technology of choice. When thermal properties are an issue, yet solubility in a volatile organic solvent is acceptable, spray drying is often the selected approach. Challenges for technology selection exist when compounds possess unacceptable thermal properties for HME and poor solubility in suitable organic solvents. It is in this area where KinetiSol® provides considerable value. As a non-solvent process that can rapidly solubilize high melting point APIs in molten polymers, KinetiSol® is able to satisfy unmet needs in this area of the PWS compound space. KinetiSol® also supplements HME and spray drying in other areas when molecules are thermally labile or unstable in organic solution, for example. Additionally, KinetiSol® may be an alternative to spray drying when cost of goods or the availability of high-volume manufacturing capacity is an issue.

With the increasing challenges of PWS drugs, unmet needs are emerging that cannot be satisfied with only HME and spray drying. KinetiSol® offers unique and viable solutions to these issues and consequently adds substantial value to the field of ASD technologies. This article presents the unique attributes of the KinetiSol® process and its novel applications to the production of ASD systems by reviewing the current literature on the technology.

PWS drugs with very high melting points (>200° C.) are emerging from drug discovery with greater frequency in recent years. High melting point compounds present significant challenges to thermal processing for the production of ASD systems, namely carrier degradation and amorphous drug-loading limitations. When processing these compounds by HME, extrusion temperatures are limited by the onset of thermal degradation of the polymer carrier, and thus processing temperatures can be 100° C. or more below the melting point of the drug. In this processing regime, the drug must be solubilized by the molten polymer in order to achieve an amorphous composition. Solubilization of the API in the molten polymer is controlled by dissolution kinetics, and typically, the time required to solubilize the drug fraction is proportional to its melting point. This requires the use of a carrier that is capable of fully and rapidly solubilizing the API at temperatures well below its melting point, which limits the range of excipients available for formulation design. For very high melting point compounds, it can be extremely difficult or impossible to achieve target drug loads without extended residence time distributions, greater mechanical energy input, and/or high temperatures, all of which can lead to degradation of the API and/or polymer carrier.

The advantages of thermokinetic compounding such as KinetiSol® for thermal processing of high melting point compounds are two-fold: (1) the energy input typically renders crystalline compounds amorphous well below their melting points and (2) the process's high shear rates significantly accelerate solubilization kinetics of APIs in molten polymers. Consequently, KinetiSol® can render high melting point APIs amorphous at temperatures well within the thermal limits of most pharmaceutical polymers. Additionally, the rapid solubilization kinetics provided by KinetiSol allow for the achievement of high amorphous drug loadings. In this section, case studies are reviewed, illustrating the applicability of KinetiSol® to the production of ASD systems with high melting point compounds.

In addition, many of the polymers commonly used in solid dispersion systems present challenges for fusion-based processing with respect to thermal sensitivity, high viscosity, or both. Often, plasticizers and other processing aids are required to reduce polymer glass transition temperatures to enable thermal processing of viscous systems or to facilitate thermal processing at lower temperatures. Because KinetiSol® is not torque-limited, highly viscous polymer systems are no issue to process without plasticizers. Furthermore, very brief processing times at elevated temperature (typically less than 5 seconds) reduce thermal stress on the polymer, and hence reduction of process temperatures by incorporating a plasticizer is not required. Even further, the unique modes of shear induced by KinetiSol® facilitate amorphous composition formation well below the melting point of most drugs, thus processing temperatures are typically lower with KinetiSol® than HME. The elimination of plasticizers leads to increased composite glass transition temperatures and improved physical stability, in most cases.

### III. Active Pharmaceutical Ingredients (APIs)

### A. Weakly Basic APIs

Weakly basic APIs refer to any weakly basic new chemical entity, drug or active pharmaceutical ingredient that does not completely release a hydroxide ion in an aqueous medium, including bases (organic and inorganic), drug salts, polymorphs, stereoisomers, solvates, esters and mixtures thereof. In one embodiment of the present invention, a weakly basic compound of the composition refers to a compound having a measured or calculated pKb of less than 14 (equivalent to a pKa of >0). In another embodiment, a weakly basic compound of the composition refers to a compound having a pKb of less than 14, a pH dependent solubility at physiological pH, and a decrease in solubility with increasing pH. In another embodiment, the weakly basic compound of the composition is a compound having a pKb of 1.0 to 14.0, a pH-dependent solubility at physiological pH conditions (1.0 to 8.0) and a low solubility at about pH 8.0-12.0. As another example, weakly basic compounds refer to compounds having a solubility of less than 1 mg/mL at pH 10.0-14.0. In another embodiment, the weakly basic compound comprises a compound having at least one basic functional group. In another example, a weakly basic compound refers to a compound having a pKb of less than 14 and a solubility of less than 1 mg/mL at pH 12.0. In another embodiment, a weakly acidic compound refers to a compound having a pKb of less than 14 and at least one basic functional group. In another embodiment, a weakly basic compound refers to a compound having a pKb of less than 14, a solubility of less than 1 mg/mL at pH 12.0, and at least one basic functional group. In another embodiment, a weakly basic compound refers to a compound that must first undergo hydrolysis before releasing a hydroxide ion.

Such active drug molecules include, but are not limited to, analgesics, antihypertensives, anxiolytics, anticoagulants, anticonvulsants, antidiabetics, hypoglycemic agents, decongestants, antihistamines, anti-inflammatory agents, antitussives, antineoplastics, beta-blockers, antirheumatics, anti-inflammatory agents, antipsychotics, pro-cognitive agents, anti-atherosclerotic agents, antiobesity agents, yang-tonics, anti-infective agents, hypnotics, anti-Parkinsonism agents, anti-Alzheimer's disease agents, antidepressants, antiviral agents, phosphatase inhibitors, cholesteryl ester transfer protein inhibitors, CNS (central nervous system) stimulants, dopamine receptor agonists, anti-emetics, gastrointestinal agents, psychotic disorders, opioid receptor agonists, opioid receptor antagonists, antiepileptics, histamine H-antagonists₂Antagonists, antiasthmatic agents, smooth muscle relaxants and skeletal muscle relaxants. Examples of analgesics include rofecoxib, celecoxib, morphine, codeine, oxycodone, hydrocodone, heroin, meperidine, tramadol, buprenorphine; antihypertensives include prazosin, nifedipine, lercanidipine, amlodipine besylate, tramozosin and doxazosin; examples of anxiolytics include hydroxyzine hydrochloride, lorazepam, buspirone, pramipepam, linaloon, meprobamate, oxazepam, trifluoperazine hydrochloride, potassium chlorooxalate, diazepam; examples of anticoagulants include abciximab, eptifibatide, tirofiban, lamifiban, clopidogrel, ticlopidine, dicoumarin, heparin, and warfarin; examples of antiepileptic drugs include phenobarbital, oxazepine, clonazepam, chlordiazepoxide, diazepam, midazolam, lorazepam, felbamateCarbamazepine, oxcarbazepine, vigabatrin, pregabalin, tiagabine, topiramate, gabapentin, pregabalin, ethionine, phenytoin, mephenytoin, phenytoin, diethyltolune, trimethadione, dioxanone, beclomethamide, pramipedone, bravaracetam, levetiracetam, cetrimide, ethosuximide, phensuximide, mesuximide, acetazolamide, thiothiazine, methazolamide, zonisamide, lamotrigine, phenylbutylurea, phenylacetyl urea, valproamide and pennogamide; examples of antidiabetic agents include repaglinide, nateglinide, metformin, phenformin, rosiglitazone, pioglitazone, troglitazone, miglitol, acarbose, exenatide, vildagliptin and sitagliptin; examples of hypoglycemic agents include tolbutamide, acetylbenzenesulfonylcyclohexamide, tolazamide, glyburide, glimepiride, gliclazide, glipizide, and chlorpropamide; examples of decongestants include pseudoephedrine, phenylephrine, and oxymetazoline; examples of antihistaminic agents include mepyramine, antazoline, diphenhydramine, carbinoxamine, doxylamine, clemastine, dimenhydrinate, pheniramine, chlorpheniramine, levochlorpheniramine, brompheniramine, triprolidine, phendimetrazine, clorazine, hydroxyzine, chlorpheniramine, promethazine, isobutylazine, cyproheptadine, azatadine, and ketotifen; examples of antitussives include dextromethorphan, noscapine, morphine, and codeine; examples of the antitumor agent include chlorambucil, lomustine, tobrozole and echinomycin; examples of anti-inflammatory agents include betamethasone, hydroprednisone, aspirin, piroxicam, valdecoxib, carprofen, celecoxib, flurbiprofen and (+)-N-{4-[3-(4-fluorophenoxy) phenoxy-]-2-cyclopropyl-1-en}-N-hydroxyurea; examples of the antitumor agent include actinomycin, dactinomycin, doxorubicin, daunorubicin, epirubicin, bleomycin, plicamycin, and mitomycin; examples of beta-blockers include alprenolol, carteolol, levobunolol, mepindolol, metipranolol, nadolol, oxprenolol, penbutolol, pindolol, propranolol, sotalol, timolol, acebutolol, atenolol, betaxololBisoprolol, esmolol, metoprolol, nebivolol, carvedilol, celiprolol, labetalol, and butaxamine; examples of antirheumatic agents include adalimumab, azathioprine, chloroquine, hydroxychloroquine, cyclosporine, D-penicillamine, etanercept, disodium aurothioate, auranofin, inflixib, leflunomide, methotrexate, minocycline, sulfasalazine; examples of anti-inflammatory agents include steroidal and non-steroidal anti-inflammatory drugs such as hydrocortisone, prednisone, prednisolone, methylprednisolone, dexamethasone, betamethasone, triamcinolone acetonide, beclomethasone, aldosterone, acetaminophen, amoxicillin, benorilate, diflunisal, famesamine, diclofenac, aceclofenac, acemetacin, bromfenac, etodolac, indomethacin, naproxone, sulindac, toluoylpicolinic acid, carprofen, ketorolac, mefenamic acid, phenylbutazone, apazone, analgin, oxybuprazone, sulpirenone, piroxicam, lornoxicam, meloxicam, tenoxicam, celecoxib, etoxib, lumiracoxib, parecoxib, rofecoxib, valdecoxib, and nimesulide; examples of antipsychotics include iloperidone, clozapine, olanzapine, sulpirtine hydrochloride, fluspirilene, risperidone, and pentafluridol; examples of pro-cognitive drugs include ampakine; examples of anti-atherosclerotic, cardiovascular and/or cholesterol lowering agents include atorvastatin calcium, cerivastatin, fluvastatin, lovastatin, mevastatin, pitavastatin, pravastatin, rosuvastatin and simvastatin; examples of anti-obesity agents include levoamphetamine, dexfenfluramine, fenfluramine, phentermine, orlistat, acarbose and rimonabant; examples of the yang-invigorating agent include sildenafil and sildenafil citrate; examples of anti-infective agents include antibacterial, antiviral, antiprotozoal, anthelmintic, and antifungal agents including carbenicillin indan sodium, bazocillin hydrochloride, acebamomycin, doxycycline hydrochloride, ampicillin, penicillin G, azithromycin, oxytetracycline, minocycline, erythromycin, clarithromycin, spiramycin, acyclovir, nelfinavir, ribavirin, benzalkonium chloride, chlorhexidine, econazole, troconazole, fluconazoleConazole, voriconazole, griseofulvin, metronidazole, thiabendazole, oxfendazole, morantel, sulfamethoxazole; examples of hypnotics include alphaxalone and etomidate; examples of anti-parkinson agents include levodopa, bromocriptine, pramipexole, ropinirole, pergoline, and selegiline; anticholinergics such as dipheny, benztropine mesylate, prilidine, biperiden and diethylpromazine; antihistamines such as diphenhydramine and orthanimine; and amantadine; examples of anti-alzheimer's disease agents include donepezil, rivastigmine, galantamine, tacrine; examples of antibacterial agents include minocycline, rifampin, erythromycin, nafcillin, cefazolin, imipenem, aztreonam, gentamycin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, terafloxacin, tosufloxacin, clinafloxacin, sulbactam, clavulanic acid, amphotericin B, fluconazole, itraconazole, ketoconazole, nystatin; examples of antidepressants include isoxazole hydrazide, phenelzine, phencyclyamine; examples of antiviral agents include zidovudine (AZT), didanosine (dideoxyinosine, ddl), stavudine (d4T), zalcitabine (dideoxycytidine, ddC), nevirapine, lamivudine (yipingwei, 3TC), saquinavir (indinavir), ritonavir (norvir), indinavir (cannelure), delavirdine (delavirdine); examples of phosphatase inhibitors include [R—(R S).]]-5-chloro-N-[2-hydroxy-3-{methoxymethylamino}-3-oxo-1-(phenylmethyl) propyl-1H-indole-2-carboxamide and 5-chloro-1H-indole-2-carboxylic acid [(1S)-benzyl-(2R)-hydroxy-3-((3R,4S)-dihydroxy-1-pyrrolidinyl-)-3-propoxypropoxy]An amide; examples of cholesteryl ester transfer protein inhibitors include [2R,4S]]4-[(3, 5-bistrifluoromethyl-benzyl)-carbomethoxy-amino]-2-ethyl-6-trifluoromethyl-3, 4-dihydro-2H-quinoline-1-carboxylic acid ethyl ester, [2R,4S]4-[acetyl-(3, 5-bistrifluoromethyl-benzyl)-amino]-2-ethyl-6-trifluoromethyl-3, 4-dihydro-2H-quinoline-1-carboxylic acid isopropyl ester, [2R,4S]4-[(3, 5-bistrifluoromethyl-benzyl)-carbomethoxy-amino]-2-ethoxypropene-6-trifluoromethyl-3, 4-dihydro-2H-quinoline-1-carboxylic acid isopropyl ester; examples of CNS (central nervous system) stimulants include caffeine and methylphenidate; examples of dopamine receptor agonists include cabergoline and pramipexole; examples of antiemetics include dolasetron, granisetron, ondansetron, tropisetron, palonosetron, domperidone, droperidol, dimenhydrinate, haloperidol, chlorpromazine, promethazine, prochlorperazine, metoclopramide, and aripride; examples of gastrointestinal agents include loperamide and cisapride; examples of the antipsychotic agent include chlorpromazine, thioridazine, prochlorperazine, haloperidol, alprazolam, amitriptyline, bupropion, buspirone, diazepin, citalopram, clozapine, diazepam, fluoxetine, fluphenazine, fluvoxamine, hydroxyzine, cloquinethazine, loxyprexate, mirtazapine, molindone, nefazodone, nortriptyline, olanzapine, paroxetine, phenelzine, quetiapine, risperidone, sertraline, meperidine, phencyclamate, phencyclamine, trazodone, venlafaxine, and ziprasidone; examples of opioid receptor agonists include hydromorphone, fentanyl, methadone, morphine, oxycodone, and oxymorphone; examples of opioid receptor antagonists include naltrexone; examples of antiepileptic drugs include sodium valproate, nitrazepam, phenytoin; histamine H₂. Examples of antagonists include famotidine, nizatidine, cimetidine, ranitidine; examples of antiasthmatic agents include albuterol, montelukast sodium; examples of smooth muscle relaxants include nicorandil, iloperidone, and clonazepam; and examples of skeletal muscle relaxants include diazepam, lorazepam, baclofen, carisoprodol, chlorzoxazone, cyclobenzaprine, dantrolene, metaxalone, orthotolylhydramine, pancuronium, tizanidine, dicyclomine, clonidine, and gabapentin. The drugs indicated above include the free forms of the drugs and pharmaceutically acceptable salts, solvates, esters and prodrugs thereof.

Examples of a weakly basic drugs include BI 639667, ciprofloxacin, mitoxantrone, epirubicin, daunorubicin, doxorubicin, vincristine, vinblastine, lidocaine, chlorpromazine, dibucaine, propranolol, timolol, quinidine, pilocarpine, physostigmine, dopamine, serotonin, imipramine, diphenhydramine, quinine, chloroquine, quinacrine, ritonavir, itraconazole, posaconazole, nevirapine, aprepitant, albendazole, mebendazole, amprenavir, abiraterone, saquinavir, rifabutin, anthracyclines, vinca alkaloids, lamivudine, zalcitabine, didanosine, efavirenz, zidovudine, nelfinavir, indinavir, chloroquine, azathioprine, atazanavir, amiodarone, terfenadine, tamoxifen, velpatasvir, elbasvir, and codeine, pharmaceutically acceptable salts thereof, and combinations thereof.

### B. Weakly Acidic APIs

Weakly acidic APIs refer to any weakly acidic new chemical entity, drug or active pharmaceutical ingredient that does not completely donate a proton in an aqueous medium, including acids, drug salts, polymorphs, stereoisomers, solvates, esters and mixtures thereof. In one embodiment of the present invention, a weakly acidic compound of the composition refers to a compound having a measured or calculated pKa of less than 14. In another embodiment, a weakly acidic compound of the composition refers to a compound having a pKa of less than 14, a pH dependent solubility at physiological pH, and a decrease in solubility with decreasing pH. In another embodiment, the weakly acidic compound of the composition is a compound having a pKa of 0.0 to 10.0, a pH-dependent solubility at physiological pH conditions (1.0 to 8.0) and a low solubility at about pH1.0 to 2.0. As another example, weakly acidic compounds refer to compounds having a solubility of less than 1 mg/mL at pH 1.0-2.0. In another embodiment, the weakly acidic compound comprises a compound having at least one acidic functional group. In another example, a weakly acidic compound refers to a compound having a pKa of less than 14 and a solubility of less than 1 mg/mL at pH 1.2. In another embodiment, a weakly acidic compound refers to a compound having a pKa of less than 14 and at least one acidic functional group. In another embodiment, a weakly acidic compound refers to a compound having a pKa of less than 14, a solubility of less than 1 mg/mL at pH 1.2, and at least one acidic functional group. Representative of such active pharmaceutical molecules include, but are not limited to, acetaminophen, acetaminosalol, acetazolamide, tretinoin, acrivastine, ampicillin, arbutin, azelaic acid, benzoyl peroxide, caffeic acid, chlorothiazide, chlorpropamide, ciclopirox, ciprofloxacin, cromolyn, ethacrynic acid, ferulic acid, furosemide, hydroquinone, ibuprofen, kojic acid, methotrexate, penicillamine, penicillin, pentobarbital, phenobarbital, phenytoin, perindopril, propylthiouracil, rabeprazole, retinoic acid, risedronic acid, salicylic acid, sulfacetamide, sulfabenzene, sulfabenzoyl, sulfabromopyrimidine, sulfachlorpyridazine, sulfaoxetine, sulfadoxine, sulfaguanidine, sulfalene, sulfamethoxazole, sulfapyrazine, sulfadiazine, sulfasalazine, sulfisothiazole, sulfathiazole, sulfafurazol, sulfadiazine, sulfasalazine, Theophylline, lipoic acid, 6, 8-dimercaptooctanoic acid (dihydrolipoic acid), tolbutamide, triclosan, imidazole acrylic acid, ursodeoxycholic acid, and warfarin. The drugs indicated above include the free forms of the drugs and pharmaceutically acceptable salts, solvates, esters and prodrugs thereof.

### C. Delivery

A variety of administration routes are available for delivering APIs to a patient in need. The particular route selected will depend upon the particular drug selected, the weight and age of the patient, and the dosage required for therapeutic effect. The pharmaceutical compositions may conveniently be presented in unit dosage form. APIs suitable for use in accordance with the present disclosure, and its pharmaceutically acceptable salts, derivatives, analogs, prodrugs, and solvates thereof, can be administered alone, but will generally be administered in admixture with a suitable pharmaceutical excipient, adjuvant, diluent, or carrier selected with regard to the intended route of administration and standard pharmaceutical practice, and can in certain instances be administered with one or more additional API(s), preferably in the same unit dosage form.

An API may be used in a variety of application modalities, including oral delivery as tablets, capsules or suspensions; pulmonary and nasal delivery; topical delivery as emulsions, ointments or creams; transdermal delivery; and parenteral delivery as suspensions, microemulsions or depot. As used herein, the term “parenteral” includes subcutaneous, intravenous, intramuscular, or infusion routes of administration.

### D. Excipients

The excipients that may be used in the presently disclosed compositions, while potentially having some activity in their own right, are generally defined for this application as compounds that enhance the efficiency and/or efficacy of a API. It is also possible to have more than one API in a given formulation.

Any pharmaceutically acceptable excipient known to those of skill in the art may be used to produce the composites and compositions disclosed herein. Examples of excipients for use with the present disclosure include, but are not limited to, e.g., lactose, glucose, starch, calcium carbonate, kaoline, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium or sodium croscarmellose, a mixture of starch and lactose, sucrose, butter, hydrogenated oil, a mixture of a quaternary ammonium base and sodium lauryl sulfate, glycerine and starch, lactose, bentonite, colloidal silicic acid, talc, and stearates.

Excipients may be used to enhance the efficacy and efficiency of the API. Additional non-limiting examples of compounds that can be included are binders, carriers, cryoprotectants, lyoprotectants, fillers, stabilizers, protease inhibitors, antioxidants, bioavailability enhancers and absorption enhancers. The excipients may be chosen to modify the intended function of the active ingredient by improving flow, or bioavailability, or to control or delay the release of the API. Specific nonlimiting examples include sucrose, trehaolose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphadityl choline, glycolic acid and salts, deoxycholic acid and salts, sodium fusidate, cyclodextrins, Labrasol®, polyvinyl alcohols, polyvinyl pyrrolidones and tyloxapol.

Polymer carriers or thermal binders that may or may not require a plasticizer include, for example, Eudragit® RS PO, Eudragit® S100, Kollidon® SR (poly(vinyl acetate)-co-poly(vinylpyrrolidone) copolymer), Ethocel® (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), sodium carboxymethyl-cellulose (CMC), dimethylaminoethyl methacrylate-methacrylic acid ester copolymer, ethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit® L-30-D (MA-EA, 1:1), Eudragit® L100-55 (MA-EA, 1:1), Eudragit® EPO (poly(butyl methacylate-co-(2-dimethylaminoethyl) methacrylate-co-methyl methacrylate) 1:2:1), hydroxypropylmethylcellulose acetate succinate (HPMCAS), Coateric® (PVAP), Aquateric® (CAP), and AQUACOAT® (HPMCAS), Soluplus® (polyvinyl caprolactam-polyvinyl acetate-polyethylene glycol graft copolymer, BASF), Luvitec® K 30 (polyvinylpyrrolidone, PVP), Kollidon® (polyvinylpyrrolidone, PVP), polycaprolactone, starches, pectins; polysaccharides such as tragacanth, gum arabic, guar gum, and xanthan gum.

Water soluble polymers include Soluplus®, methylcellulose, ethylcellulose, hydroxymethylcellulose, hydroxyethylcellulose, hydroxypropylcellulose, hydroxybutylcellulose, hydroxyethyl methylcellulose, hydroxypropyl methylcellulose, carboxymethylcellulose, hydroxypropyl methylcellulose acetate succinate, cellulose acetate phthalate, cellulose acetate trimellate, hydroxypropyl methylcellulose phthalate, cellulose acetate butyrate, polyvinylpyrrolidones, polyacrylates, polymethacrylates, polyvinyl alcohols, polyethylene glycols, polyvinyl acetate polyvinylpyrrolidone copolymers, polyethylene oxides, dimethylaminoethyl methacrylate-methacrylic acid ester copolymers, ethylacrylate-methylmethacrylate copolymers, poly(methyacrylate ethylacrylate) copolymers, poly(methacrylate methylmethacrylate) copolymers, starches, pectins, polysaccharides, gum arabic, guar gum, and xanthan gum.

The carrier may also contain various functional excipients, such as an antioxidant, super-disintegrant, surfactant including amphiphilic molecules, wetting agent, stabilizing agent, retardant, similar functional excipient, or combination thereof, plasticizers including citrate esters, polyethylene glycols, PG, triacetin, diethylphthalate, castor oil, and others known to those or ordinary skill in the art. Extruded material may also include an acidifying agent, adsorbent, alkalizing agent, buffering agent, colorant, flavorant, sweetening agent, diluent, opaquant, complexing agent, fragrance, preservative or a combination thereof.

Compositions with enhanced solubility may comprise a mixture of an API and an additive that enhances the solubility of the API. Examples of such additives include but are not limited to surfactants, polymer carriers, pharmaceutical carriers, thermal binders or other excipients. A particular example may be a mixture of an API with a surfactant or surfactants, an API with a polymer or polymers, or an API with a combination of a surfactant and polymer carrier or surfactants and polymer carriers.

Surfactants that can be used in the disclosed compositions to enhance solubility have been previously presented. Particular examples of such surfactants include but are not limited to sodium dodecyl sulfate, dioctyl docusate sodium, Tween 80, Span 20, Cremophor® EL or Vitamin E TPGS. Polymer carriers that can be used in the disclosed composition to enhance solubility have been previously presented. Particular examples of such polymer carriers include but are not limited to Soluplus®, Eudragit® L100-55, Eudragit® EPO, Kollidon® VA 64, Luvitec®. K 30, Kollidon®, AQOAT®-HF, and AQOAT®-LF. The composition of the present disclosure can thus be any combination of one or more of the APIs, zero, one or more of surfactants or zero, one or more of polymers presented herein.

Solubility can be indicated by peak solubility, which is the highest concentration reached of a species of interest over time during a solubility experiment conducted in a specified medium. The enhanced solubility can be represented as the ratio of peak solubility of the agent in a pharmaceutical composition of the present disclosure compared to peak solubility of the reference standard agent under the same conditions. Preferable, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

### IV. Examples

It will be understood that particular embodiments described herein are shown by way of illustration and not as limitations of the disclosure. The principal features of this disclosure can be employed in various embodiments without departing from the scope of the disclosure. All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

### Example 0—Rationale

Specifically, this disclosure pertains to the improvement of the bioavailability of amorphous drug loading of poorly water-soluble drugs exhibiting pH dependent solubility, due to being weak acids or weak bases, by the inclusion of strong counter bases or acids respectively. The addition of a small amount (w/w) of approximately 5% NaOH improves the drug loading of an ASD comprising telmisartan (weak acid) and Soluplus® from ˜5% maximum to at least 30% (maximum of 50%) maximum amorphous API. This improvement is not unique to NaOH but is a function of the concentration of [OH—] which is released by the basic salt when the polymer Soluplus® is made molten by the extruder. At 5% NaOH, the molar ratio of telmisartan:NaOH is 0.466:1 but the mass ratio of telmisartan:[OH—] (ignoring the contribution to mass by Na) is 14:1.

Experiments so far confirm that the ability to produce high drug load amorphous API is a function of the amount (w/w) of the base, the strength of the base (strong/low pKb or weak/high pKb), and also the water solubility of the base. Weak bases and a strong base with poor water solubility (e.g., <10 g/100 mL) were not sufficient to render a 30% telmisartan mixture amorphous at tested conditions, while 5% of two strong bases with high water solubility (e.g., >10 g/100 mL) NaOH and KOH rendered these mixtures amorphous. A third water-soluble, strong base, LiOH has a substantially lower pKb compared to the other two strong bases and caused visible degradation of the polymer suggesting a maximum base strength that can be used for this purpose.

### Example 1—Methods

Materials. Soluplus® was donated by BASF Chemical Company (Florham Park, NJ, USA. Telmisartan, USP (TEL) was purchased from Shenzhen Nexconn Pharmatechs LTD. (Shenzhen, China). All other chemicals used were ACS grade. All bases were milled in an IKA tube mill control (IKA-Werke, Staugen, Germany) set at 20,000 RPM for 30 seconds comprised of two 15 second pulsesimmediately prior to addition to physical mixtures. Hot-melt Extrusion (HME). HME was performed using a MiniHaake laboratory scale extruder. Physical mixtures were prepared by weighing components into a plastic container and then thoroughly mixing for 10 minutes. These were fed manually into the extruder during extrusion. Screw speed was held constant at 100 RPM while temperature was held constant, depending on the experiment, between 180 C and 200 C. Extrudate was air-cooled then milled and placed in scintillation vials in a desiccator prior to further analysis. Pure Soluplus® was fed in-between runs to wash out prior physical mixtures.

High-Performance Liquid Chromatography (HPLC). TEL concentrations were measured using a Waters HPLC system (Waters Corporation, Milford, MA) with a UV detector (Waters Corporation, Milford, MA). A C18 5×20 mm, 5 μm column (Thermo Scientific, Waltham, MA) was used. UV absorbance was measured at 220 nm. Injections of 10 uL were done by autosampler. A mobile phase consisting of 0.05M sodium dihydrogen phosphate buffer (pH 3.6) and acetonitrile was eluted using at a 30:70 ratio at 1.2 mL/min. The column was kept at room temperature. The retention time of TEL was 6 minutes.

Modulated Differential Scanning Calorimetry (mDSC). A mDSC (DSC 2920, TA Instruments, New Castle, Delaware) was utilized to determine thermal properties of extrudates and physical mixtures. Approximately 5-10 mcg of material was accurately weighed into standard aluminum pans which were then crimped. Samples were equilibrated at 50 C for 5 minutes and then heated from 50 C to 300 C with a heating ramp of 5 C/min with a 1 C/minute modulation program. Dry nitrogen gas was used to purge the DSC cell at 50 mL/min. TA Universal Analysis 2000 software was used for data processing.

Powder X-ray diffraction (PXRD). A Rigaku Miniflex600 II (Rigaku Americas, The Woodlands, Texas) instrument was used to determine the presence of crystalline or amorphous drug. The instrument was equipped with a Cu-Ka radiation source generated at 40 kV and 15 mA. A 2-theta angle was set to 5-35° with a step size of 0.020 and a scan rate of 2° per minute.

Polarized Light Microscopy (PLM). PLM was used to detect the presence or absence of birefringence in extrudate to determine crystalline content. Samples were prepared by lightly spreading milled material or placing extrudate directly onto a glass slide. An Olympus BX60 microscope (Olympus Corp., Center Valley, PA) with an Insight QE camera (Diagnostic Instruments, Inc., Sterling Heights, MI) was used for visual observation. Spot Advance Software (Diagnostic Instruments, Inc.) captured images.

Dissolution Study. A Hanson SR8PLUS dissolution apparatus (Hanson Research Co., Chatsworth, CA) and paddles were used to perform non-sink dissolution experiments. For all experiments, the paddle speed was set to 100 rpm and the temperature was set to 37+/−0.5° C. Each vessel contained 150 mL of pH 6.5 buffered FaSSIF (0.1N Na₃PO₄) solution that was preheated to 37 C before the addition of sample. To achieve a theoretical maximum of 50 mcg/mL of TEL in each vessel, 15 mg of either 50:7:43 TEL:KOH:Soluplus PM or ASD (7.5 mg of TEL equivalent) were added to the vessel. Extrudate was milled and the particle size was controlled by sieving. Samples for HPLC were collected at 5, 10, 15, and 30 minutes, as well as at 1 hour, 2 hours, and 20 hours. At each time point, samples were drawn up and filtered through a 0.22 μm PES filter and the 0.66 mL of the filtrate was diluted with 0.33 mL of MeOH before final analysis.

### Example 2

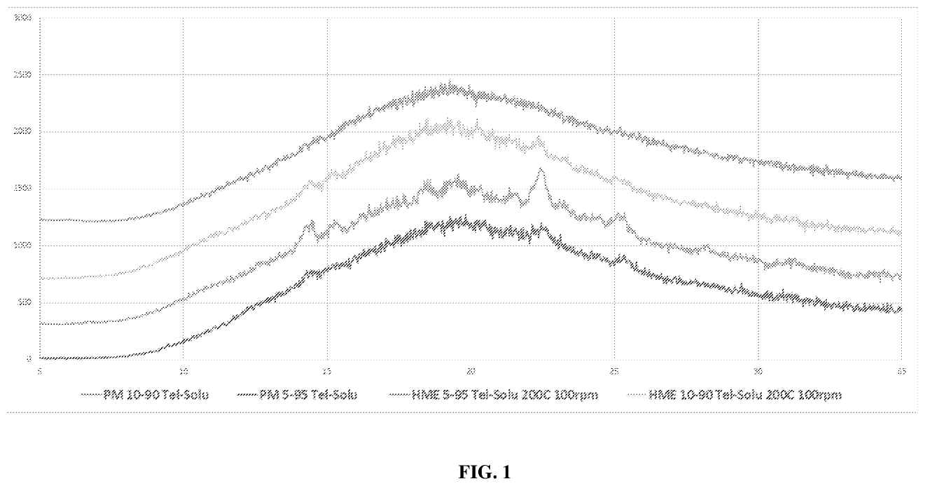

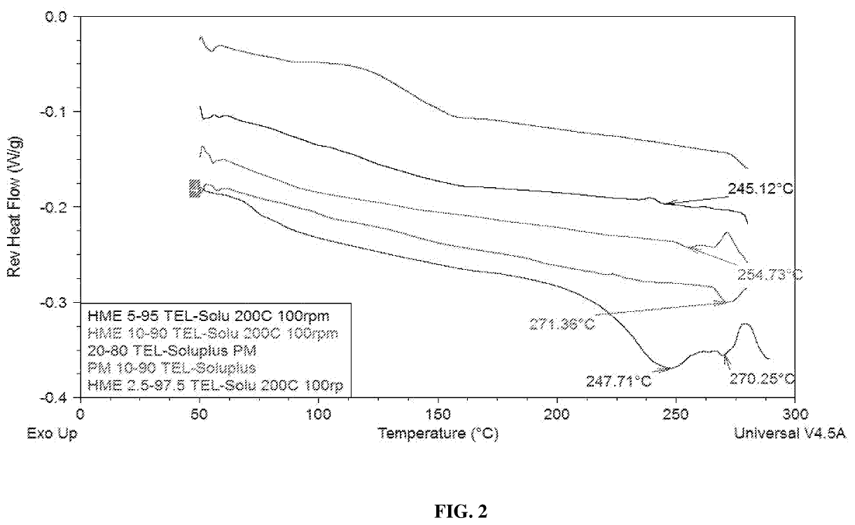

This example examined the maximum drug load of telmisartan in Soluplus® achievable by extrusion with no additional excipients. Physical mixtures of Soluplus® containing 2.5%, 5%, and 10% TEL w/w were extruded according to the methods above with the temperature set to 200° C. Milled extrudate and physical mixture were examined by PXRD (FIG. 1) and DSC (FIG. 2). The 2.5% and 5% were amorphous by DSC as evidenced by a lack of an endotherm between 220° C.-260° C. as well as no melting endotherm at the melting point of TEL of approximately 270° C. In contrast, the 10% w/w TEL extrudate has an endotherm at approximately 254° C. that indicates the dissolution of TEL in Soluplus® below its melting point. The 20% w/w TEL physical mixture, used as a control, shows both a small melting endotherm at Telmisartan's melting point (˜270° C.) and a gradual endotherm starting at 220° C. and ending near 260° C. that is hypothesized to be caused by the gradual dissolution of TEL in Soluplus® over this temperature range.

PXRD results for the 5% w/w TEL extrudate had a lack of crystalline peaks suggesting conversion of amorphous drug, although it should be noted that the limit of detection (LoD) of crystalline drug in polymer is approximately 5% w/w and so cannot be conclusively relied on. In agreement with this general trend, both the 2.5% w/w physical mixture and extrudate had no significant crystalline peaks (data not shown). In contrast, the 10% w/w TEL extrudate had characteristic crystalline telmisartan peaks at approximately 140 and 23° 2-theta indicating that crystalline material was still present.

### Example 3

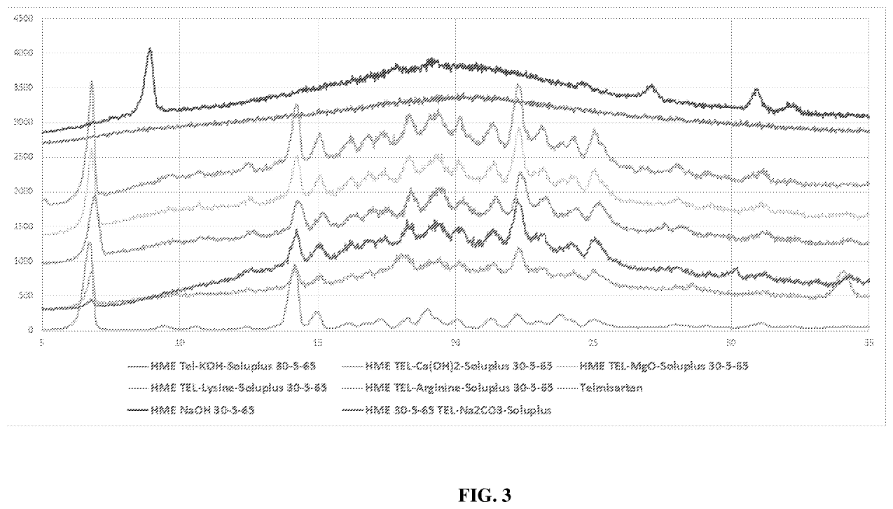

This example examined the ability of different bases to increase the maximum drug loading of TEL when 5% w/w is incorporated into the physical mixture. Physical mixtures of 30:5:65 w/w TEL:Base:Soluplus® with various bases (detailed in Table 1) were prepared and extruded at 180° C. according to the methods described above. Extrudate was milled and examined by PXRD (FIG. 3). Extrudates prepared with the strong bases that also had high (e.g., >10 g/100 mL) water solubilities (LiOH, NaOH, and KOH) did not have characteristic TEL peaks and were considered to be amorphous. The other extrudates with bases that were entirely weak (arginine, lysine), weak but convert to strong base during processing (Na₂CO₃->NaOH), or strong but have low water solubility (e.g., <10 g/100 mL) (Ca(OH)₂), all failed to convert the TEL amorphous as evidenced by TEL peaks in the PXRD.

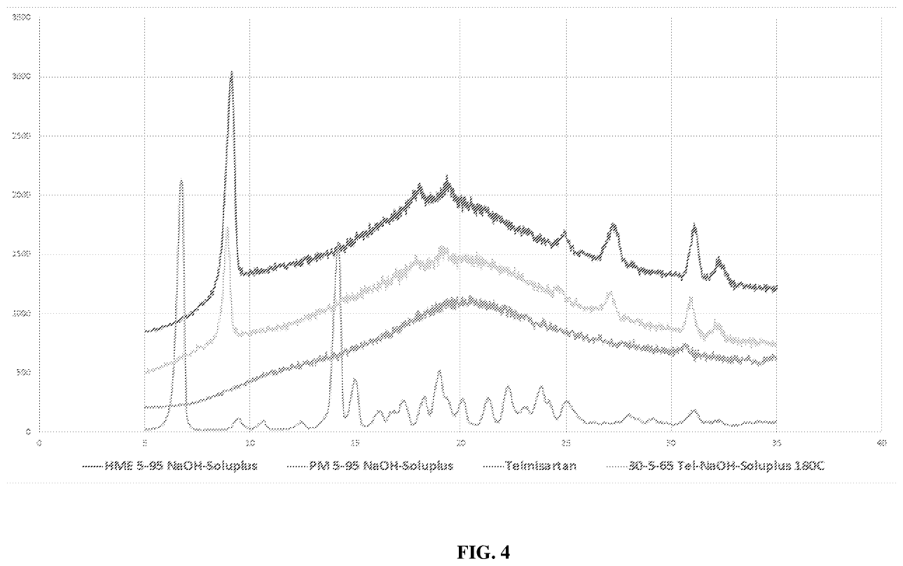

The extrudate containing NaOH had several crystalline peaks that were not characteristic of TEL. To determine the cause of these peaks, Soluplus® with 5% w/w NaOH was extruded (without TEL) and the extrudate and physical mixture were examined by PXRD (FIG. 4). All crystalline peaks in the 30:5:65 TEL:NaOH:Soluplus® extrudate matched those in the 5:95 NaOH:Soluplus® extrudate and confirmed that crystalline TEL was converted amorphous.

TABLE 1
Identities and characteristics of various bases screened for
ability to increase drug loading of Telmisartan during HME.

| | | | | MgO | | | | Sodium |
|---|---|---|---|---|---|---|---|---|
| Base | LiOH | NaOH | KOH | (Mg(OH)₂) | Ca(OH)₂ | L-lysine | L-arginine | carbonate |
| pK_{b} | −0.36 | 0.2 | 0.5 | N/A (1.2) | 2.4 | 3.5 | 1.5 | 3.7 |
| Weak/Strong | Strong | Strong | Strong | (N/A) | Strong | Weak | Weak | Weak |
| | | | | (borderline) | | | (borderline) | |
| Solubility (g/100 mL) | 12.8 | 418 | 121 | <1 | <1 | 150 | 15 | 34 |
| Room Temperature | | | | | | | | |

### Example 4

The maximum drug loading of TEL in Soluplus® using weak bases, weak bases that become strong bases during processing, or strong bases with low (e.g., <10 g/100 mL) water solubility was examined using HME. Physical mixtures containing 10% base w/w and between 10-30% TEL were prepared with Soluplus® and extruded at 200° C. according to the method described above.

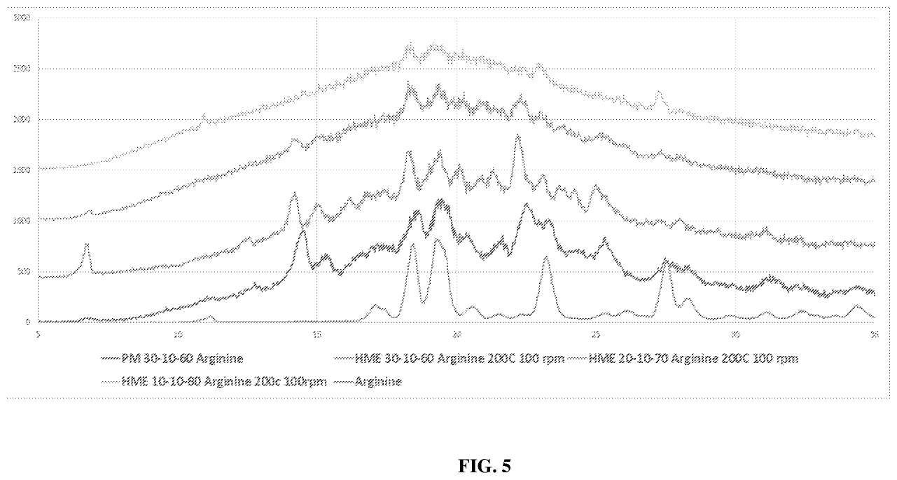

Arginine was chosen as an exemplary weak base and extrudate containing 10, 20, and 30% TEL was examined by PXRD (FIG. 5) and compared to a physical mixture containing 30:10:60 TEL:Arginine:Soluplus®. Peaks indicating the presence of TEL were present at concentrations above 10%, indicating that a 10% drug loading was the highest achievable under these conditions.

Na₂CO₃+H₂O→2NaOH+CO₂ Equation 1

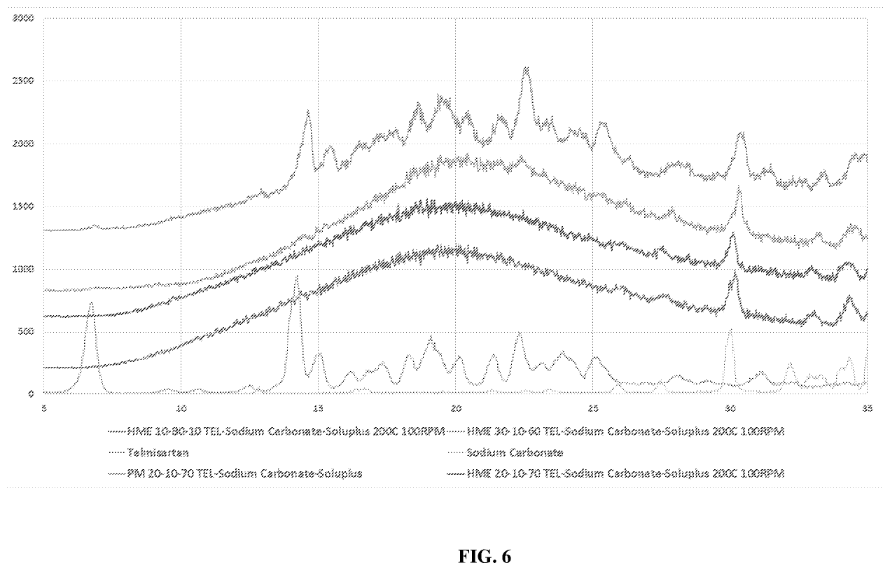

Sodium carbonate (Na₂CO₃) was chosen as an example of a weak base that during extrusion as it becomes a strong base as described in Equation 1. As CO₂ escapes during extrusion, the reaction of Na₂CO₃->NaOH moves towards completion according to Le Chatelier's Principle. Additionally, the CO₂ escaping during extrusion visually creates a more porous extrudate, which can provide performance benefits. Extrudate containing 10, 20, and 30% TEL was examined by PXRD (FIG. 6) and compared to a physical mixture of 20:10:70 TEL:Na₂CO₃:Soluplus®. Peaks indicating the presence of TEL were present at concentrations above 20%, indicating that a 20% drug loading was the highest achievable under these conditions.

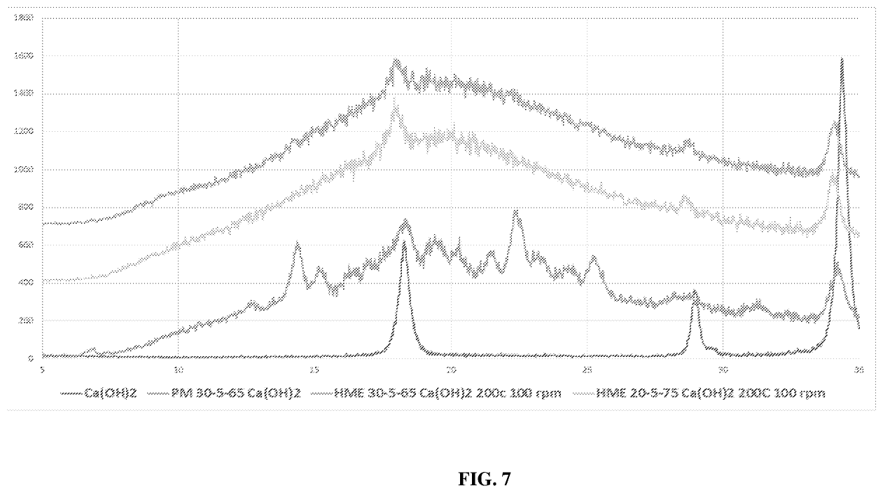

Calcium carbonate (Ca(OH)₂) was chosen as an example of a strong base with low water solubility. Extrudate containing 20, and 30% TEL was examined by PXRD (FIG. 7) and compared to a physical mixture of 30:5:70 TEL:Ca(OH)₂:Soluplus®. Peaks indicating the presence of TEL were present at concentrations above 20%, indicating that a 20% drug loading was the highest achievable under these conditions.

### Example 5

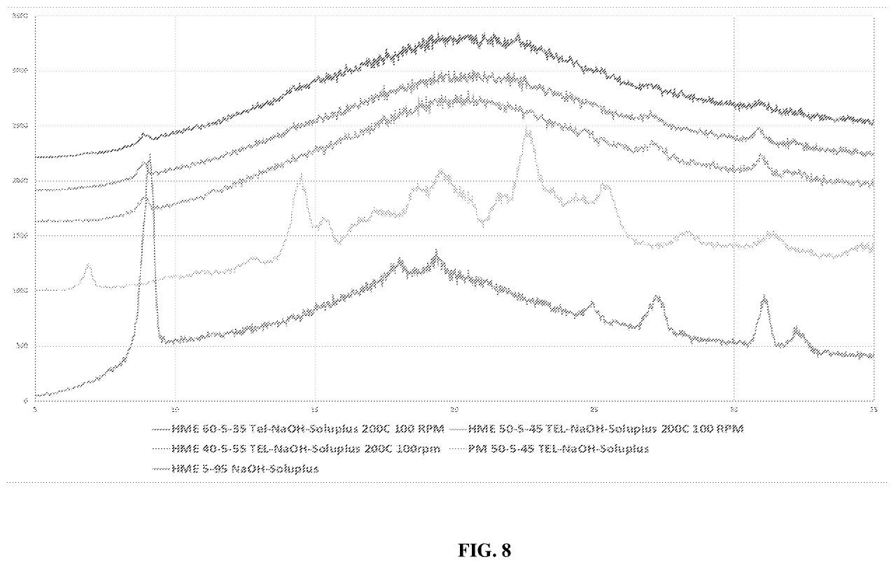

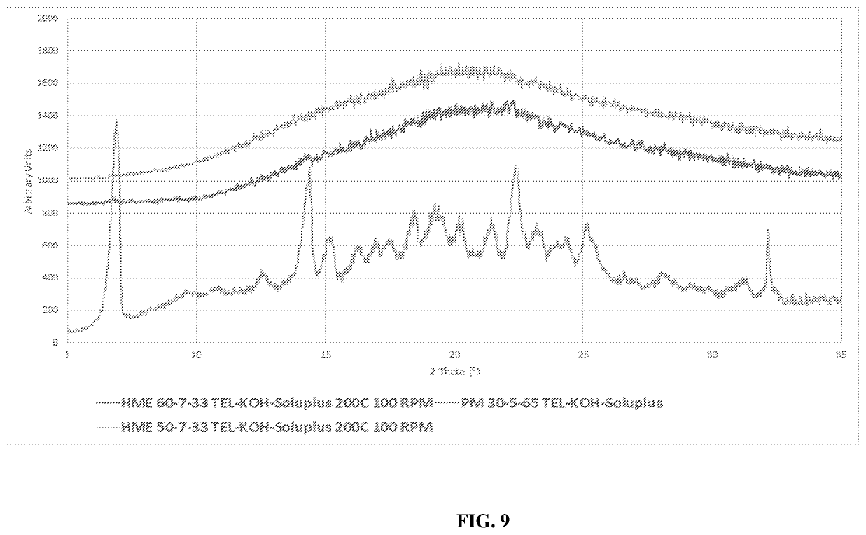

The maximum drug loading of TEL in Soluplus® using strong, water soluble bases was examined. Sodium hydroxide (NaOH) and potassium hydroxide (KOH) were chosen as examples bases. To control for mass percent of (OH—) in the base, 5% and 7% of NaOH and KOH, respectively were added to physical mixtures containing up to 60% TEL. Both these w/w amounts yield approximately 2.12% (OH—) by weight. Extrudate containing up to 60% TEL was examined by PXRD (FIGS. 8 and 9) and compared to a physical mixture containing the respective bases as well as the extrudate of 5:95 NaOH:Soluplus® for NaOH. Peaks indicating the presence of TEL were present at concentrations above 50%, indicating that a 50% drug loading was the highest achievable under these conditions.

### Example 6

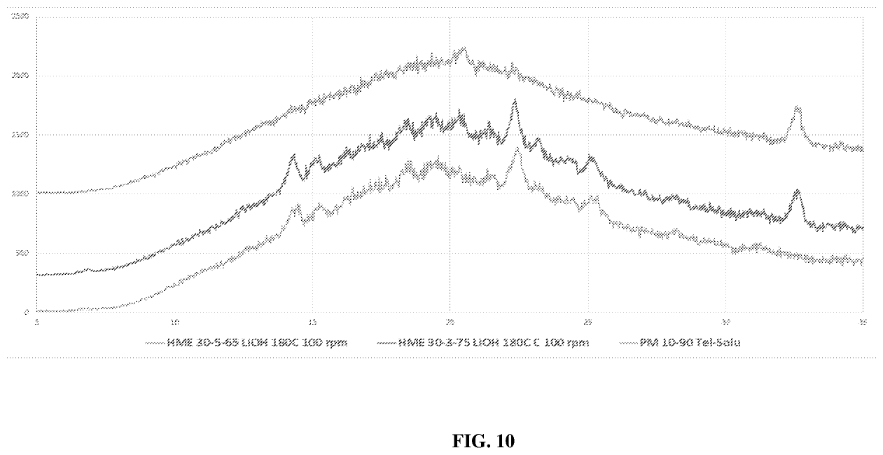

The effect of the concentration of base was examined by varying the concentration of LiOH in a 30% TEL physical mixture in Soluplus®. In addition, the suitability of bases with a pKb<0 was examined for improving the miscibility of TEL in Soluplus®. Both 3% and 5% LiOH containing extrudates were examined by PXRD (FIG. 10). While the 5% LiOH containing extrudate did not have peaks characteristic of crystalline TEL, the 3% LiOH extrudate did. This suggests that there is a minimum concentration of strong base that is suitable for this purpose. In addition, the extrudates of both mixtures were deemed to not be pharmaceutically acceptable based on physical appearance; both had brown fragments indicating that either Soluplus® or TEL was being degraded when in close contact with this base. No other TEL:Base:Soluplus® combination, besides for a 40-10-50 TEL-NaOH-Soluplus®, hypothesized to result in unsuitably high hydroxide concentration, tested was similarly unsuitable. LiOH was the base with the lowest reported pKb of less than 0 and so was deemed unsuitable for this purpose due to this.

### Example 7

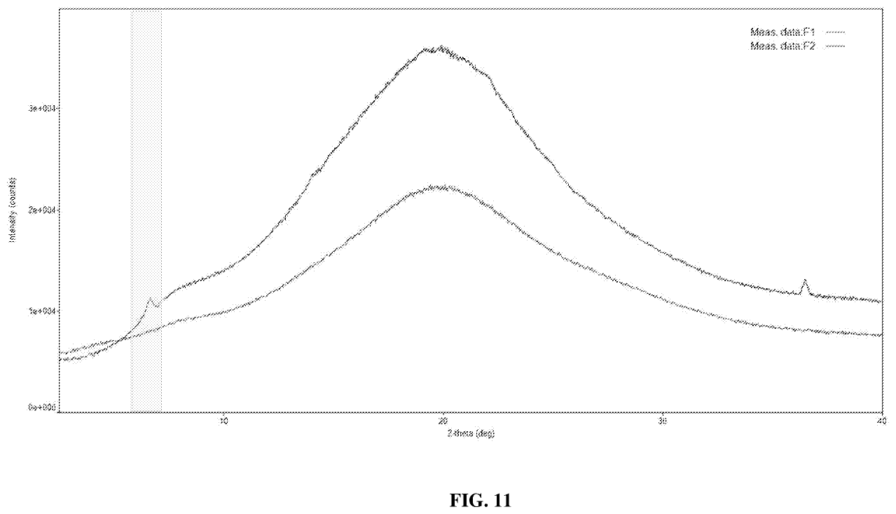

In this example, the use of high energy mixing by KinetiSol dispersing to transform crystalline TEL (weak-acid) amorphous in the presence of a strong base and in the absence of a strong base is reported. Composition F1 comprised Telmisartan:Potassium hydroxide:Soluplus:magnesium stearate (30:5:64.5:0.5). Composition F2 comprised Telmisartan:Soluplus:magnesium stearate (30:69.5:0.5). Magnesium stearate is used in these formulations as a lubricant to aid the high energy mixing process. Similar processing conditions for composition F1 and F2 were selected, with the only difference being the presence or absence of the strong base in the final compositions. 15 grams of compositions F1 and F2 were processed at 5500 RPM for 30 seconds with an ejection temperature of 180 C, if the ejection temperature was not reached after 30 seconds, 6500 RPM was used to reach the ejection temperature of 180° C. For composition F1, ejecting at 180° C. rendered composition F1 entirely amorphous by PXRD. For composition F2, ejecting at 180° C. incompletely rendered the composition amorphous and crystallinity attributed to Telmisartan at 6.8° was detected during PXRD analysis as shown in FIG. 11.

### Example 8

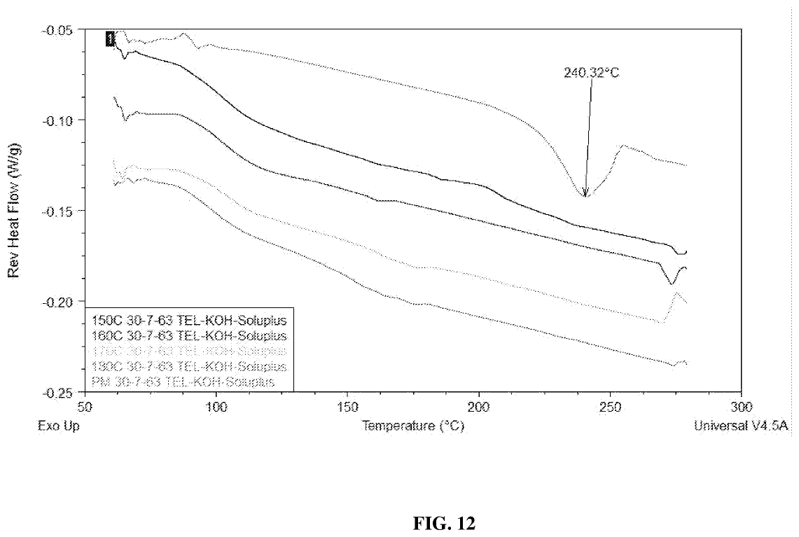

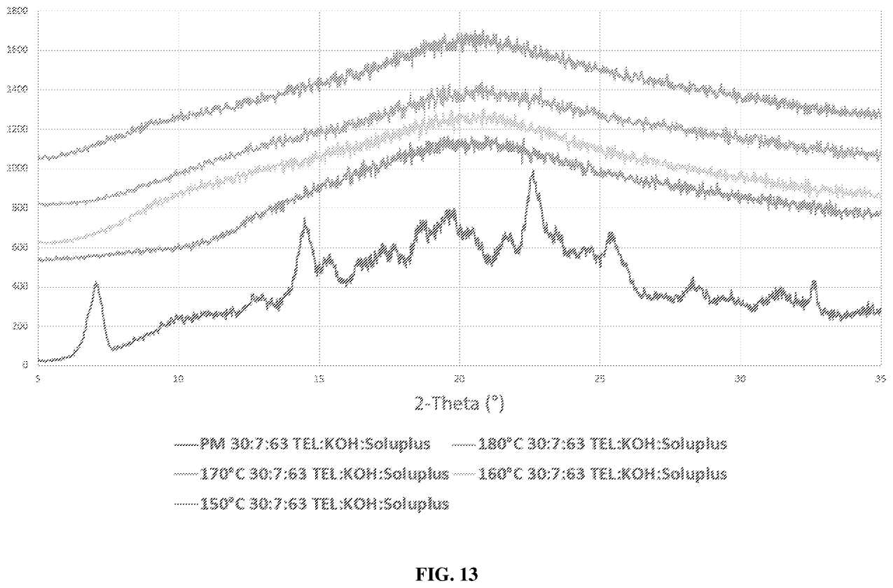

The ability to produce ASD containing strong base at temperatures substantially below the melting point of the weakly acidic API was examined. The API chosen was TEL and has a melting point of approximately 271° C. A physical mixture consisting of 30:7:63 w/w TEL:KOH:Soluplus was extruded at 150° C., 160° C., 170° C. and 180° C. at 100 RPM. Each of these extrudates were milled and examined by mDSC (FIG. 12) and PXRD (FIG. 13). Samples examined by mDSC were considered to contain crystalline TEL if there was an endotherm obtained between 220° C.-260° C. and by PXRD if crystalline peaks were present at 6.8, 13, 15, and 23° 2-theta. All four samples were amorphous by these techniques, indicating that with the addition of a comparatively small amount (w/w) of a strong base, that a 30% w/w TEL ASD can be successfully produced at temperatures more than 120° C. below the melting point. This is in contrast to the failure to produce a 10% w/w TEL ASD at 200° C. without the addition of any bases as described in Example 2.

### Example 9

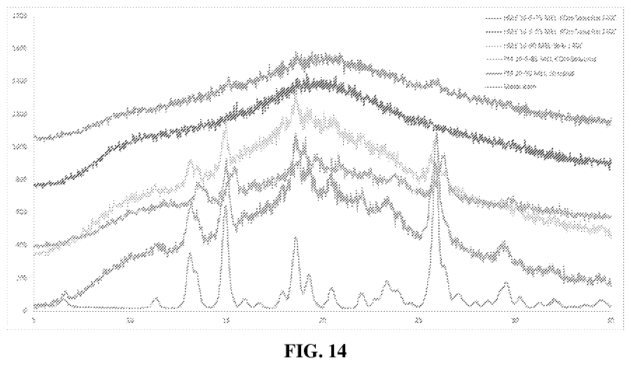

The ability to reduce the processing temperature required to render a weakly acidic compound meloxicam amorphous by HME was examined. Meloxicam has a melting temperature of approximately 255° C. Physical mixtures of meloxicam and Soluplus with or without 5% w/w KOH were prepared and extruded at 140° C. and 100 RPM on a MiniHaake extruder. The 10% w/w meloxicam and Soluplus extrudate without KOH had substantial crystallinity by XRD while the equivalent one with KOH had no crystalline peaks, indicating that the meloxicam had been rendered completely amorphous. See FIG. 14. When these same mixtures were processed at 150° C., the extrudate was a visibly degraded grey color. This shows that the incorporation of KOH allows for the reduction of processing temperature to approximately 115° C. below the melting point of the compound as well as below its degradation temperature. In addition, the 20% meloxicam and Soluplus with KOH extrudate had only trace crystallinity, less than the 10% meloxicam without KOH, indicating that the KOH aids in increasing the concentration of amorphous meloxicam that can be loaded into the amorphous solid dispersion.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. References

The following references, to the extent that they provide exemplary procedural or other details supplementary to those set forth herein, are specifically incorporated herein by reference.
(1) Friedl T, Schepky G, inventors; Boehringer Ingelheim International GmbH, assignee. Method for preparation of substantially amorphous telmisartan. Canadian Patent C A 2651604. 2013 Apr. 9.
(2) Evans R C, Bochmann E S, Kyeremateng S O, Gryczke A, Wagner K G. Holistic QbD approach for hot-melt extrusion process design space evaluation: Linking materials science, experimentation and process modeling. European Journal of Pharmaceutics and Biopharmaceutics. 2019 Aug. 1; 141:149-60.
(3) Haser A, Huang S, Listro T, White D, Zhang F. An approach for chemical stability during melt extrusion of a drug substance with a high melting point. International journal of pharmaceutics. 2017 May 30; 524(1-2):55-64.
(4) Rossi D T, Li B, Mccall J P, inventors; Mylan Inc, assignee. Stabilized tolterodine tartrate formulations. U.S. Pat. No. 8,778,397. 2014 Jul. 15.
(5) Crowley M, Keen J, Koleng J, Zhang F, inventors; Auxilium U S Holdings, LLC, assignee. Stabilized Compositions containing alkaline labile drugs. U.S. Pat. No. 9,867,786 B2. Jan. 16, 2018.
(6) Parikh T, Serajuddin A T. Development of fast-dissolving amorphous solid dispersion of itraconazole by melt extrusion of its mixture with weak organic carboxylic acid and polymer. Pharmaceutical research. 2018 July; 35(7):1-0.
(7) Patel N G, Serajuddin A T. Development of FDM 3D-printed tablets with rapid drug release, high drug-polymer miscibility and reduced printing temperature by applying the acid-base supersolubilization (ABS) principle. International Journal of Pharmaceutics. 2021 Mar. 26:120524.
(8) Giri B R, Kwon J, Vo A Q, Bhagurkar A M, Bandari S, Kim D W. Hot-Melt Extruded Amorphous Solid Dispersion for Solubility, Stability, and Bioavailability Enhancement of Telmisartan. Pharmaceuticals. 2021 January; 14(1):73.
(9) Haser A, Cao T, Lubach J, Listro T, Acquarulo L, Zhang F. Melt extrusion vs. spray drying: The effect of processing methods on crystalline content of naproxen-povidone formulations. European Journal of Pharmaceutical Sciences. 2017 May 1; 102:115-25.
(10) Maa Y F, Nguyen P A, Andya J D, Dasovich N, Sweeney T D, Shire S J, Hsu C C. Effect of spray drying and subsequent processing conditions on residual moisture content and physical/biochemical stability of protein inhalation powders. Pharmaceutical research. 1998 May; 15(5):768-75.
(11) Almotairy A, Almutairi M, Althobaiti A, Alyahya M, Sarabu S, Alzahrani A, et al. Effect of pH modifiers on the solubility, dissolution rate, and stability of telmisartan solid dispersions produced by hot-melt extrusion technology. J Drug Deliv Sci Technol [Internet]. 2021; 102674.

What is claimed is:

1. A method of preparing a pharmaceutical composition comprising an amorphous solid dispersion (ASD) of a poorly water-soluble active pharmaceutical ingredient (API), wherein said API is a weak base or weak acid, comprising:
(a) providing an API that is a weak base or weak acid;
(b) contacting said API with a polymeric carrier and:
(i) a strong base when said API is a weak acid; or
(ii) a strong acid when said API is a weak base; and
(c) processing the mixture of step (b) to produce an ASD.

2. The method of claim 1, wherein the API exhibits a high melting point, is thermally sensitive, is shear sensitive, and/or exhibits pH dependent solubility with thermal and/or high energy processing.

3. The method of claims 1-2, wherein processing comprises a high energy mixing process, such as high energy fusion process (e.g., KinetiSol® Dispersing), or a thermal process, such as hot melt extrusion.

4. The method of claims 1-3, wherein the water solubility of the API is less than 1 mg/mL when measured using high pressure liquid chromatography (HPLC) at 20° C.

5. The method of claims 1-3, wherein the API is telmisartan, itraconazole, meloxicam, diclofenac, or atazanavir.

6. The method of claims 1-5, wherein the strong base is NaOH, KOH, Ca(OH)₂, or LiOH.

7. The method of claims 1-5, wherein the strong base is sodium hydroxide or potassium hydroxide, which is formed in situ from sodium carbonate or potassium carbonate, respectively.

8. The method of claim 7, wherein the sodium hydroxide is produced from Na₂CO₃ from CO₂ escape during extrusion of the mixture.

9. The method according to any one of claim 1-8, wherein steps (b) and (c) occur simultaneously.

10. The method of claims 1-5, wherein the strong base is a water-soluble strong base.

11. The method of claims 1-5, wherein the strong acid is hydrochloric acid, chloric acid, sulfuric acid, hydrobromic acid, nitric acid, or P-toluene sulfonic acid.

12. The method of claims 1-5, wherein the strong acid is a water-soluble strong acid.

13. The method of claims 1-12, wherein the mixture of step (b) comprises 0.1-15% (w/w) strong base or strong acid and/or wherein the polymer is present in said pharmaceutical composition at about 1% to 50% (w/w).

14. The method of claims 1-13, wherein the polymeric carrier is water soluble, e.g., Soluplus®, methylcellulose, ethylcellulose, hydroxymethylcellulose, hydroxyethylcellulose, hydroxypropylcellulose, hydroxybutylcellulose, hydroxyethyl methylcellulose, hydroxypropyl methylcellulose, carboxymethylcellulose, hydroxypropyl methylcellulose acetate succinate, cellulose acetate phthalate, cellulose acetate trimellate, hydroxypropyl methylcellulose phthalate, cellulose acetate butyrate, polyvinylpyrrolidones, polyacrylates, polymethacrylates, polyvinyl alcohols, polyethylene glycols, polyvinyl acetate polyvinylpyrrolidone copolymers, polyethylene oxides, dimethylaminoethyl methacrylate-methacrylic acid ester copolymers, ethylacrylate-methylmethacrylate copolymers, poly(methyacrylate ethylacrylate) copolymers, poly(methacrylate methylmethacrylate) copolymers, starches, pectins, polysaccharides, gum arabic, guar gum, and xanthan gum.

15. The method of claims 1-14, wherein the pharmaceutical composition does not contain a plasticizer and/or does not contain a processing agent.

16. The method of claims 1-15, wherein the pharmaceutical composition has an increase in drug loading compared to a pharmaceutical composition prepared by a comparable method without said strong acid or strong base.

17. The method of claim 16, wherein the increase in drug loading is about 2.5% to about 50%.

18. The method of claims 1-17, wherein said pharmaceutical composition comprises less than about 1% degradation products of said API.

19. The method of claims 1-18, wherein the ratio of API to polymeric carrier is about 3:2, 5:4, 1:1, 2:3, 1:2, 1:3, 1:4, or 1:5.

20. The method of claims 1-19, wherein step (b) is performed at a temperature of about 100° C., about 125° C., about 150° C., about 175° C., about 200° C., about 220° C., or about 100° C. to about 220° C.

21. The method of claims 1-20, wherein said pharmaceutical composition comprises a second API, such as composition that contains two or more API are used to treat hypertension (e.g., ACE inhibitors such as lisinopril, enalapril, benazepril, captopril; beta-blockers such as metoprolol, carvedilol, esmolol, nebivolol; calcium channel blockers such as diltiazem, verapamil, amlodipine, nifedipine; diuretics including thiazide diuretics such as hydrochlorothiazide and chlorthalidone or furosemide, spironolactone, bumetanide, torsemide or others), diabetes (e.g., metformin; SGLT2 inhibitors including empagliflozin, dapagliflozin, canagliflozin; DDP4 inhibitors including sitagliptin, saxagliptin, linagliptin; GLP-1 agonists including dulaglutide, exenatide, semaglutide; and others), or cancer (e.g., chlorambucil, lomustine, tobrozole and echinomycin).

22. The method of claims 1-21, wherein said pharmaceutical composition comprises a surfactant.

23. A pharmaceutical composition prepared according to the method of claims 1-22.
